SEQUENCE LISTING (38 sequences)

SEQ ID NO: 1 - RNA, 87 nt, Artificial Sequence
  synthetic hsa-let-7f-1 precursor miRNA
ucagagugagguaguagauuguauaguugugggguagugauuuuacccuguucaggagauaacuauacaaucuauugccuucccuga

SEQ ID NO: 2 - RNA, 22 nt, Artificial Sequence
  synthetic hsa-let-7f-1 and hsa-let-7f-2 mature miRNA
ugagguaguagauuguauaguu

SEQ ID NO: 3 - RNA, 83 nt, Artificial Sequence
  synthetic hsa-let-7f-2 precursor miRNA
ugugggaugagguaguagauuguauaguuuuagggucauaccccaucuuggagauaacuauacagucuacugucuuucccacg

SEQ ID NO: 4 - RNA, 84 nt, Artificial Sequence
  synthetic hsa-let-7g precursor miRNA
aggcugagguaguaguuuguacaguuugagggucuaugauaccacccgguacaggagauaacuguacaggccacugccuugcca

SEQ ID NO: 5 - RNA, 22 nt, Artificial Sequence
  synthetic hsa-let-7g mature miRNA
ugagguaguaguuuguacaguu

SEQ ID NO: 6 - RNA, 84 nt, Artificial Sequence; identical to SEQ ID NO: 4
  synthetic hsa-let-7g* precursor miRNA

SEQ ID NO: 7 - RNA, 21 nt, Artificial Sequence
  synthetic hsa-let-7g* mature miRNA
cuguacaggccacugccuugc

SEQ ID NO: 8 - RNA, 83 nt, Artificial Sequence
  synthetic hsa-miR-15a precursor miRNA
ccuuggaguaaaguagcagcacauaaugguuuguggauuuugaaaaggugcaggccauauugugcugccucaaaaauacaagg

SEQ ID NO: 9 - RNA, 22 nt, Artificial Sequence
  synthetic hsa-miR-15a mature miRNA
uagcagcacauaaugguuugug

SEQ ID NO: 10 - RNA, 98 nt, Artificial Sequence
  synthetic hsa-miR-15b precursor miRNA
uugaggccuuaaaguacuguagcagcacaucaugguuuacaugcuacagucaagaugcgaaucauuauuugcugcucuagaaauuuaaggaaauucau

SEQ ID NO: 11 - RNA, 22 nt, Artificial Sequence
  synthetic hsa-miR-15b mature miRNA
uagcagcacaucaugguuuaca

SEQ ID NO: 12 - RNA, 89 nt, Artificial Sequence
  synthetic hsa-miR-16-1 precursor miRNA
gucagcagugccuuagcagcacguaaauauuggcguuaagauucuaaaauuaucuccaguauuaacugugcugcugaaguaagguugac

SEQ ID NO: 13 - RNA, 22 nt, Artificial Sequence
  synthetic hsa-miR-16-1 and hsa-miR-16-2 mature miRNA
uagcagcacguaaauauuggcg

SEQ ID NO: 14 - RNA, 81 nt, Artificial Sequence
  synthetic hsa-miR-16-2 precursor miRNA
guuccacucuagcagcacguaaauauuggcguagugaaauauauauuaaacaccaauauuacugugcugcuuuagugugac

SEQ ID NO: 15 - RNA, 84 nt, Artificial Sequence
  synthetic hsa-miR-17 precursor miRNA
gucagaauaaugucaaagugcuuacagugcagguagugauaugugcaucuacugcagugaaggcacuuguagcauuauggugac

SEQ ID NO: 16 - RNA, 23 nt, Artificial Sequence
  synthetic hsa-miR-17 mature miRNA
caaagugcuuacagugcagguag

SEQ ID NO: 17 - RNA, 72 nt, Artificial Sequence
  synthetic hsa-miR-21 precursor miRNA
ugucggguagcuuaucagacugauguugacuguugaaucucauggcaacaccagucgaugggcugucugaca

SEQ ID NO: 18 - RNA, 21 nt, Artificial Sequence
  synthetic hsa-miR-21 mature miRNA
caacaccagucgaugggcugu

SEQ ID NO: 19 - RNA, 86 nt, Artificial Sequence
  synthetic hsa-miR-28-3p precursor miRNA
gguccuugcccucaaggagcucacagucuauugaguuaccuuucugacuuucccacuagauugugagcuccuggagggcaggcacu

SEQ ID NO: 20 - RNA, 22 nt, Artificial Sequence
  synthetic hsa-miR-28-3p mature miRNA
cacuagauugugagcuccugga

SEQ ID NO: 21 - RNA, 88 nt, Artificial Sequence
  synthetic hsa-miR-30b precursor miRNA
accaaguuucaguucauguaaacauccuacacucagcuguaauacauggauuggcugggagguggauguuuacuucagcugacuugga

SEQ ID NO: 22 - RNA, 22 nt, Artificial Sequence
  synthetic hsa-miR-30b mature miRNA
uguaaacauccuacacucagcu

SEQ ID NO: 23 - RNA, 81 nt, Artificial Sequence
  synthetic hsa-miR-106a precursor miRNA
ccuuggccauguaaaagugcuuacagugcagguagcuuuuugagaucuacugcaauguaagcacuucuuacauuaccaugg

SEQ ID NO: 24 - RNA, 23 nt, Artificial Sequence
  synthetic hsa-miR-106a mature miRNA
aaaagugcuuacagugcagguag

SEQ ID NO: 25 - RNA, 110 nt, Artificial Sequence
  synthetic hsa-miR-182 precursor miRNA
gagcugcuugccuccccccguuuuuggcaaugguagaacucacacuggugagguaacaggauccggugguucuagacuugccaacuauggggcgaggacucagccggcac

SEQ ID NO: 26 - RNA, 24 nt, Artificial Sequence
  synthetic hsa-miR-182 mature miRNA
uuuggcaaugguagaacucacacu

SEQ ID NO: 27 - RNA, 110 nt, Artificial Sequence
  synthetic hsa-miR-223 precursor miRNA
ccuggccuccugcagugccacgcuccguguauuugacaagcugaguuggacacuccaugugguagagugucaguuugucaaauaccccaagugcggcacaugcuuaccag

SEQ ID NO: 28 - RNA, 22 nt, Artificial Sequence
  synthetic hsa-miR-223 mature miRNA
cguguauuugacaagcugaguu

SEQ ID NO: 29 - RNA, 94 nt, Artificial Sequence
  synthetic hsa-miR-575 precursor miRNA
aauucagcccugccacuggcuuaugucaugaccuugggcuacucaggcugucugcacaaugagccaguuggacaggagcagugccacucaacuc

SEQ ID NO: 30 - RNA, 19 nt, Artificial Sequence
  synthetic hsa-miR-575 mature miRNA
gagccaguuggacaggagc

SEQ ID NO: 31 - RNA, 97 nt, Artificial Sequence
  synthetic hsa-miR-649 precursor miRNA
ggccuagccaaauacuguauuuuugaucgacauuugguugaaaaauaucuauguauuaguaaaccuguguuguucaagaguccacuguguuuugcug

SEQ ID NO: 32 - RNA, 22 nt, Artificial Sequence
  synthetic hsa-miR-649 mature miRNA
aaaccuguguuguucaagaguc

SEQ ID NO: 33 - RNA, 98 nt, Artificial Sequence
  synthetic hsa-miR-652 precursor miRNA
acgaauggcuaugcacugcacaacccuaggagagggugccauucacauagacuauaauugaauggcgccacuaggguugugcagugcacaaccuacac

SEQ ID NO: 34 - RNA, 21 nt, Artificial Sequence
  synthetic hsa-miR-652 mature miRNA
aauggcgccacuaggguugug

SEQ ID NO: 35 - RNA, 69 nt, Artificial Sequence
  synthetic hsa-miR-1229 precursor miRNA
guggguaggguuugggggagagcgugggcugggguucagggacacccucucaccacugcccucccacag

SEQ ID NO: 36 - RNA, 23 nt, Artificial Sequence
  synthetic hsa-miR-1229 mature miRNA
cucucaccacugcccucccacag

SEQ ID NO: 37 - RNA, 90 nt, Artificial Sequence
  synthetic hsa-miR-1287 precursor miRNA
guugugcuguccaggugcuggaucagugguucgagucugagccuuuaaaagccacucuagccacagaugcagugauuggagccaugacaa

SEQ ID NO: 38 - RNA, 22 nt, Artificial Sequence
  synthetic hsa-miR-1287 mature miRNA
ugcuggaucagugguucgaguc

(19) United States
(12) Patent Application Publication
(10) Pub. No.: US 2014/0179549 A1
(43) Pub. Date: Jun. 26, 2014

(54) METHODS FOR IDENTIFYING INFLAMMATORY BOWEL DISEASE PATIENTS WITH DYSPLASIA OR CANCER

(71) Applicants: BOSTON MEDICAL CENTER CORPORATION, Boston, MA (US); NESTEC S.A., Vevey (CH)

(72) Inventors: FRANCIS FARRAYE, Boston, MA (US); Fred Princen, La Jolla, CA (US)

(73) Assignee: NESTEC S.A., Vevey (CH)

(21) Appl. No.: 14/102,432

(22) Filed: Dec. 10, 2013

Related U.S. Application Data
(63) Continuation of application No. PCT/US2012/042533, filed on Jun. 14, 2012.
(60) Provisional application No. 61/497,016, filed on Jun. 14, 2011.
(60) Provisional application No. 61/551,330, filed on Oct. 25, 2011.
(60) Provisional application No. 61/565,445, filed on Nov. 30, 2011.
(60) Provisional application No. 61/648,529, filed on May 17, 2012.

(51) Int. Cl. C12Q 1/68

(52) U.S. Cl. CPC C12Q 1/6883; C12Q 1/6886
USPC 506/9

(57) ABSTRACT

The present invention provides methods for identifying IBD patients with dysplasia or cancer. In particular embodiments, the methods of the invention may comprise determining the presence or level of at least one or a panel of miRNAs in a sample obtained from an IBD patient to establish a miRNA expression profile, and comparing the miRNA expression profile with one or more pre-established model miRNA expression profiles. The present invention further provides methods for monitoring the efficacy of treatment of IBD patients with dysplasia or cancer.

### CROSS-REFERENCE TO RELATED APPLICATIONS

This application is a continuation of PCT/US2012/042533, which application claims priority to U.S. Provisional Application No. 61/497,016, filed Jun. 14, 2011, U.S. Provisional Application No. 61/551,330, filed Oct. 25, 2011, U.S. Provisional Application No. 61/565,445, filed Nov. 30, 2011, and U.S. Provisional Application No. 61/648,529, filed May 17, 2012, the disclosures of which are hereby incorporated by reference in their entirety for all purposes.

### BACKGROUND OF THE INVENTION

Patients with inflammatory bowel disease (IBD) have an increased risk of developing colorectal neoplasia including dysplasia and colorectal cancer (CRC). The standard of care in 2010 is to perform surveillance colonoscopies every 1-2 years with biopsies in patients with longstanding and extensive IBD to identify dysplasia prior to the development of cancer. However, colonoscopy is an invasive procedure and some individuals develop CRC despite undergoing repeated colonoscopic procedures.

MicroRNAs (e.g., miRNAs or miRs) are a class of small, non-coding RNAs that control gene expression by hybridizing to and triggering either translational repression or, less frequently, degradation of a messenger RNA (mRNA) target. The discovery and study of miRNAs has revealed miRNA-mediated gene regulatory mechanisms that play important roles in development and various cellular processes, such as cell differentiation, cell growth and cell death. Recent studies suggest that aberrant expression of particular miRNAs may be involved in human diseases, such as neurological disorders.

There is a need in the art for improved methods for detecting and identifying dysplasia and cancers in patients with IBD. The present invention provides novel methods of identifying miRNA expression profiles from IBD patients with and without dysplasia and cancer for diagnosis and prognosis of disease. The present invention also provides methods of using miRNA profiles isolated from patient samples to assist the clinician in identifying IBD patients with colorectal neoplasia (e.g., CRC).

### BRIEF SUMMARY OF THE INVENTION

The present invention provides methods for identifying IBD patients with dysplasia or cancer. In particular embodiments, the methods of the invention comprise determining the presence or level of at least one microRNA (e.g., miRNA or miR) in a sample such as a blood sample obtained from a patient to establish a miRNA expression profile, and comparing the miRNA expression profile with one or more pre-established model miRNA expression profiles from a control or reference standard, e.g., a normal control, a cancer control (cells, tissues, or tumors), or both. The present invention further provides methods for monitoring the efficacy of treatment of IBD patients with dysplasia or cancer, e.g., by comparing first and second miRNA expression profiles in samples obtained from patients before, during, and/or after therapy. In preferred embodiments, the at least one miRNA is a population or group or pool or panel of miRNAs.

In some aspects, the present invention provides a method for identifying an IBD patient with dysplasia or cancer, the method comprising:
(a) determining (e.g., measuring) the presence or level (e.g., expression level) of at least one or a plurality of (e.g., at least 1, 2, 3, 4, 5, 6, 7, 8, 9, 10, 11, 12, 13, 14, 15, or more) miRNAs in a sample (e.g., a blood sample) from the patient to establish a miRNA expression profile;
(b) comparing the miRNA expression profile with one or more pre-established model miRNA expression profiles from a control and/or reference standard (e.g., an IBD control sample without dysplasia or cancer, a non-IBD cancer sample, etc.); and
(c) identifying dysplasia or cancer in the IBD patient based upon the comparison in step (b).

In some embodiments, an IBD patient is identified as having or suspected of having dysplasia or cancer when at least 1, 2, 3, 4, 5, 6, 7, 8, 9, 10, 11, 12, 13, 14, 15, or more of the miRNAs in the miRNA expression profile are dysregulated (e.g., up-regulated and/or down-regulated) compared to the identical miRNAs in one or more pre-established model miRNA expression profiles. In particular embodiments, an miRNA in an IBD patient is dysregulated when there is at least a 1.5, 2, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 15, 20, 25, 30, 35, 40, 45, 50, or more fold change in expression (e.g., up-regulation or down-regulation) compared to the identical miRNA in a control and/or reference sample.

In certain embodiments, the at least one or a plurality of miRNAs is selected from the group consisting of hsa-miR-16, hsa-miR-15b, hsa-miR-15a, hsa-let-7f, hsa-miR-30b, hsa-miR-649, hsa-miR-575, hsa-miR-106a, hsa-let-7g, hsa-miR-223, hsa-miR-17, hsa-miR-652, hsa-miR-1287, hsa-miR-1229, hsa-miR-21, hsa-miR-28-3p, hsa-miR-182, hsa-let-7g*, and combinations thereof. In some instances, at least 1, 2, 3, 4, 5, 6, 7, 8, 9, 10, 11, 12, 13, 14, 15, 16, 17, or 18 of these miRNAs (alone or in combination with at least one or a plurality of other miRNAs) are determined to establish a miRNA expression profile. In particular embodiments, an IBD patient with 3 or more of these miRNAs dysregulated (e.g., up-regulated and/or down-regulated) should be suspected or identified as having dysplasia or cancer.

In one embodiment, the at least one or a plurality of miRNAs is selected from the group consisting of hsa-miR-16, hsa-miR-15b, hsa-miR-15a, hsa-let-7f, hsa-miR-30b, hsa-miR-649, hsa-miR-575, hsa-miR-106a, hsa-let-7g, hsa-miR-223, hsa-miR-17, hsa-miR-652, hsa-miR-1287, hsa-miR-1229, hsa-miR-21, and combinations thereof. In some instances, at least 1, 2, 3, 4, 5, 6, 7, 8, 9, 10, 11, 12, 13, 14, or all 15 of these miRNAs are determined to establish a miRNA expression profile. In particular embodiments, an IBD patient with 3 or more of these miRNAs down-regulated should be suspected or identified as having dysplasia or cancer.

In another embodiment, the at least one or a plurality of miRNAs is selected from the group consisting of hsa-miR-30b, hsa-miR-106a, hsa-miR-15b, hsa-miR-649, hsa-miR-16, hsa-miR-652, hsa-let-7f, hsa-let-7g, hsa-miR-1287, hsa-miR-17, hsa-miR-1229, hsa-miR-28-3p, hsa-miR-182, hsa-let-7g*, and combinations thereof. In some instances, at least 1, 2, 3, 4, 5, 6, 7, 8, 9, 10, 11, 12, 13, or all 14 of these miRNAs are determined to establish a miRNA expression profile. In particular embodiments, an IBD patient with 3 or more of these miRNAs dysregulated (e.g., up-regulated and/or down-regulated) should be suspected or identified as having dysplasia or cancer.

In other aspects, the present invention provides a method for monitoring the efficacy of treatment of an IBD patient with dysplasia or cancer, the method comprising:
(a) determining (e.g., measuring) the presence or level (e.g., expression level) of at least one or a plurality of (e.g., at least 1, 2, 3, 4, 5, 6, 7, 8, 9, 10, 11, 12, 13, 14, 15, or more) miRNAs in a first sample (e.g., a blood sample) from the patient to establish a first miRNA expression profile, wherein the first sample is obtained before treatment;
(b) determining (e.g., measuring) the presence or level (e.g., expression level) of the same miRNAs in a second sample (e.g., a blood sample) from the patient to establish a second miRNA expression profile, wherein the second sample is obtained during or after treatment;
(c) comparing the first miRNA expression profile to the second miRNA expression profile; and
(d) monitoring the efficacy of treatment for the IBD patient (e.g., whether the patient is in remission) based upon the comparison in step (c).

In certain embodiments, the method further comprises comparing the first miRNA expression profile with one or more pre-established model miRNA expression profiles from a control and/or reference standard (e.g., an IBD control sample without dysplasia or cancer, a non-IBD cancer sample, etc.).

In other embodiments, the method further comprises comparing the second miRNA expression profile with one or more pre-established model miRNA expression profiles from a control and/or reference standard (e.g., an IBD control sample without dysplasia or cancer, a non-IBD cancer sample, etc.).

In some embodiments, the treatment for the IBD patient comprises chemotherapy, surgery, or combinations thereof.

In certain other embodiments, the at least one or a plurality of miRNAs is selected from any one or a combination of the miRNAs described herein. In particular embodiments, the at least one or a plurality of miRNAs is selected from the group consisting of hsa-miR-30b, hsa-miR-106a, hsa-miR-15b, hsa-miR-649, hsa-miR-16, hsa-miR-652, hsa-let-7f, hsa-let-7g, hsa-miR-1287, hsa-miR-17, hsa-miR-1229, hsa-miR-28-3p, hsa-miR-182, hsa-let-7g*, and combinations thereof.

Other objects, features, and advantages of the present invention will be apparent to one of skill in the art from the following detailed description and figures.

### BRIEF DESCRIPTION OF THE DRAWINGS

FIG. 1 shows archived IBD samples (CD or UC) without dysplasia or cancer.

FIG. 2A shows nine control IBD samples that were negative using the miRNA assay. FIG. 2B shows three control IBD samples that were positive using the miRNA assay.

FIG. 3 shows that the set of 14 miRNAs described herein can identify IBD patients with cancer.

FIG. 4 shows that the set of 14 miRNAs described herein can identify IBD patients with flat dysplasia.

FIG. 5 shows non-IBD colon cancer and sporadic tubular adenomas in IBD.

FIG. 6 shows that the set of 14 miRNAs described herein can be used to monitor CD patients with small bowel cancer before and after treatment.

FIG. 7 shows one embodiment of a pathway analysis for the miRNAs described in Example 3.

### DETAILED DESCRIPTION OF THE INVENTION

### I. General

The present invention is based in part upon the surprising discovery that expression levels and/or patterns of blood miRNAs can be used to determine if IBD patients also suffer from dysplasia or cancer such as colorectal cancer. The miRNAs are typically isolated from whole blood or rare circulating cells such as, e.g., circulating tumor cells (CTCs), circulating endothelial cells (CECs), circulating endothelial progenitor cells (CEPCs), cancer stem cells (CSCs), disseminated tumor cells of the lymph node, and combinations thereof. The isolated miRNAs can be used to measure their expression and the expression profile can indicate the presence or absence of dysplasia or a cancer such as small bowel cancer or colorectal cancer. As such, the determination of a specific miRNA expression profiles in blood advantageously enables the prediction and identification of dysplasia or a cancer such as small bowel cancer or colorectal cancer in the patient without using an invasive procedure like colonoscopy.

### II. Definitions

As used herein, the following terms have the meanings ascribed to them unless specified otherwise.

The term “inflammatory bowel disease” or “IBD” includes gastrointestinal disorders such as, e.g., Crohn's disease (CD), ulcerative colitis (UC), and indeterminate colitis (IC). Inflammatory bowel diseases (e.g., CD, UC, and IC) are distinguished from all other disorders, syndromes, and abnormalities of the gastroenterological tract, including irritable bowel syndrome (IBS). U.S. Patent Publication No. 2008/0131439, entitled “Methods of Diagnosing Inflammatory Bowel Disease,” is incorporated herein by reference in its entirety for all purposes.

“Dysplasia” includes an abnormality in the maturation of cells within a tissue which generally involves an expansion of immature cells with a corresponding decrease in the number and location of mature cells. Dysplasia is often indicative of an early neoplastic process. The term “dysplasia” is typically used when the cellular abnormality is restricted to the originating tissue, as in the case of an early, in situ neoplasm. In certain embodiments, dysplasia is the earliest form of pre-cancerous lesion that is recognizable in a pap smear or in a biopsy by a pathologist, and can be low grade or high grade. Dysplasia is characterized by one or more (preferably all four) of the following major pathological microscopic changes: (1) anisocytosis (cells of unequal size); (2) poikilocytosis (abnormally shaped cells); (3) hyperchromatism; and/or (4) the presence of mitotic figures (an unusual number of cells which are currently dividing). In particular embodiments, the dysplasia is present in any portion of the gastrointestinal tract, including, but not limited to, the esophagous, stomach, small intestine, colon, rectum, anus, and combinations thereof.

The term “cancer” includes any member of a class of diseases characterized by the uncontrolled growth of aberrant cells. The term includes all known cancers and neoplastic conditions, whether characterized as malignant, benign, soft tissue, or solid, and cancers of all stages and grades including pre- and post-metastatic cancers. Non-limiting examples of different types of cancer include digestive and gastrointestinal cancers (e.g., colorectal cancer, small intestine (small bowel) cancer; gastrointestinal stromal tumors, gastrointestinal carcinoid tumors, colon cancer, rectal cancer, anal cancer, bile duct cancer, gastric (stomach) cancer; esophageal cancer; appendix cancer; and the like); gallbladder cancer; liver cancer; pancreatic cancer; breast cancer; lung cancer (e.g., non-small cell lung cancer); prostate cancer; ovarian cancer; renal cancer (e.g., renal cell carcinoma); cancer of the central nervous system; skin cancer; choriocarcinomas; head and neck cancers; hematological malignancies (e.g., leukemia, lymphoma); osteogenic sarcomas (e.g., Ewing sarcoma); soft tissue sarcomas (e.g., Dermatofibrosarcoma Protuberans (DFSP), rhabdomyosarcoma); other soft tissue malignancies, and papillary thyroid carcinomas. As used herein, a “tumor” comprises one or more cancerous cells.

The term “sample” includes any biological specimen obtained from an individual. Suitable samples for use in the present invention include, without limitation, whole blood, plasma, serum, saliva, urine, stool, tears, any other bodily fluid, tissue samples (e.g., biopsy), and cellular extracts thereof (e.g., red blood cellular extract). In one particular embodiment, the sample is whole blood. In another embodiment, the sample is serum. The use of samples such as serum, saliva, and urine is well known in the art (see, e.g., Hashida et al., J. Clin. Lab. Anal., 11:267-86 (1997)). One skilled in the art will appreciate that samples such as whole blood, serum, and plasma can be diluted prior to the analysis of marker levels.

The term “marker” includes any biochemical marker, serological marker, genetic marker, or other clinical or echographic characteristic that can be used in the methods of the present invention. Examples of such markers include genetic markers such as miRNAs including, but not limited to, hsa-miR-16, hsa-miR-15b, hsa-miR-15a, hsa-let-7f, hsa-miR-30b, hsa-miR-649, hsa-miR-575, hsa-miR-106a, hsa-let-7g, hsa-miR-223, hsa-miR-17, hsa-miR-652, hsa-miR-1287, hsa-miR-1229, hsa-miR-21, hsa-miR-28-3p, hsa-miR-182, hsa-let-7g*, and combinations thereof. Non-limiting examples of other markers include serological markers such as, for example, an anti-neutrophil antibody, an anti-Saccharomyces cerevisiae antibody, an antimicrobial antibody, an acute phase protein, an apolipoprotein, a defensin, a growth factor, a cytokine, a cadherin, a cellular adhesion molecule; other genetic markers such as, e.g., NOD2/CARD15; and combinations thereof. In some embodiments, the markers are utilized in combination with a statistical analysis such as an algorithm to provide a risk of developing or to identify dysplasia and/or cancer.

The term “marker profile” includes at least 1, 2, 3, 4, 5, 6, 7, 8, 9, 10, 11, 12, 13, 14, 15, 16, 17, 18, 19, 20, or more of the markers described herein, wherein the markers can be a genetic marker, a serological marker, a protein marker, and the like. In some embodiments, the marker profile together with a statistical analysis can provide physicians and caregivers valuable diagnostic and prognostic insight. In other embodiments, the marker profile with optionally a statistical analysis provides a projected response to biological therapy. An exemplary statistical analysis is a quartile score and the quartile score for each of the markers can be summed to generate a quartile sum score. In certain instances, by using one or more markers (e.g., genetic, serological, protein, etc.) in conjunction with statistical analyses, the assays described herein provide diagnostic, prognostic, and therapeutic value by identifying or predicting a risk (e.g., probability, likelihood, etc.) of developing dysplasia and/or cancer in an individual (e.g., an IBD patient) and assisting in the selection of therapy.

The term “individual,” “subject,” or “patient” typically includes humans, but also includes other animals such as, e.g., other primates, rodents, canines, felines, equines, ovines, porcines, and the like.

The terms “miRNA,” “microRNA” or “miR” are used interchangeably herein and include single-stranded RNA molecules of about 21-23 nucleotides in length, which regulate gene expression. miRNAs are encoded by genes from whose DNA they are transcribed but miRNAs are not translated into protein (non-coding RNA); instead each primary transcript (a pri-miRNA) is processed into a short stem-loop structure called a pre-miRNA and finally into a functional miRNA. Mature miRs are partially complementary to one or more messenger RNA (mRNA) molecules, and their main function is to down-regulate gene expression. Embodiments described herein include predictive, diagnostic, prognostic, and therapeutic applications.

### III. Description of the Embodiments

The present invention provides methods for determining the miRNA expression profile in IBD patients to identify the presence of or susceptibility to dysplasia or cancer. In particular embodiments, the methods of the invention comprise determining the presence or level of at least one or a panel of miRNAs in a sample such as a blood sample obtained from a patient to establish a miRNA expression profile, and then comparing the miRNA expression profile with one or more pre-established model miRNA expression profiles from a control or reference standard. The present invention also provides methods for monitoring the efficacy of treatment of IBD patients with dysplasia or cancer, e.g., by comparing first and second miRNA expression profiles in samples obtained from patients before, during, and/or after therapy.

In some aspects, the present invention provides a method for identifying an IBD patient with dysplasia or cancer, the method comprising:
(a) determining (e.g., measuring) the presence or level (e.g., expression level) of at least one or a plurality of (e.g., at least 1, 2, 3, 4, 5, 6, 7, 8, 9, 10, 11, 12, 13, 14, 15, or more) miRNAs in a sample (e.g., a blood sample) from the patient to establish a miRNA expression profile;
(b) comparing the miRNA expression profile with one or more pre-established model miRNA expression profiles from a control and/or reference standard (e.g., an IBD control sample without dysplasia or cancer, a non-IBD cancer sample such as a non-IBD colorectal cancer sample, etc.); and
(c) identifying dysplasia or cancer in the IBD patient based upon the comparison in step (b).

In some embodiments, an IBD patient is identified as having or suspected of having dysplasia or cancer when at least 1, 2, 3, 4, 5, 6, 7, 8, 9, 10, 11, 12, 13, 14, 15, or more of the miRNAs in the miRNA expression profile are dysregulated (e.g., up-regulated and/or down-regulated) compared to the identical miRNAs in one or more pre-established model miRNA expression profiles. In particular embodiments, a miRNA in an IBD patient is dysregulated when there is at least a 1.5, 2, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 15, 20, 25, 30, 35, 40, 45, 50, or more fold change in expression (e.g., up-regulation or down-regulation) compared to the identical miRNA in a control and/or reference sample. In preferred embodiments, a miRNA in the miRNA expression profile is dysregulated when there is at least about a 2-fold change in its expression level compared to the identical miRNA in the one or more pre-established model miRNA expression profiles.

In certain embodiments, the at least one or a plurality of miRNAs is selected from the group consisting of hsa-miR-16, hsa-miR-15b, hsa-miR-15a, hsa-let-7f, hsa-miR-30b, hsa-miR-649, hsa-miR-575, hsa-miR-106a, hsa-let-7g, hsa-miR-223, hsa-miR-17, hsa-miR-652, hsa-miR-1287, hsa-miR-1229, hsa-miR-21, hsa-miR-28-3p, hsa-miR-182, hsa-let-7g*, and combinations thereof. In some instances, at least 1, 2, 3, 4, 5, 6, 7, 8, 9, 10, 11, 12, 13, 14, 15, 16, 17, or 18 of these miRNAs (alone or in combination with at least one or a plurality of other miRNAs) are determined to establish a miRNA expression profile. In particular embodiments, an IBD patient with at least 1, 2, 3, 4, 5, 6, 7, 8, 9, 10, 11, 12, 13, 14, 15, 16, 17, or 18 of these miRNAs determined to be dysregulated (e.g., up-regulated and/or down-regulated) is suspected of or identified as having dysplasia or cancer.

In one embodiment, the at least one or a plurality of miRNAs is selected from the group consisting of hsa-miR-16, hsa-miR-15b, hsa-miR-15a, hsa-let-7f, hsa-miR-30b, hsa-miR-649, hsa-miR-575, hsa-miR-106a, hsa-let-7g, hsa-miR-223, hsa-miR-17, hsa-miR-652, hsa-miR-1287, hsa-miR-1229, hsa-miR-21, and combinations thereof. In some instances, at least 1, 2, 3, 4, 5, 6, 7, 8, 9, 10, 11, 12, 13, 14, or all 15 of these miRNAs are determined to establish a miRNA expression profile. In particular embodiments, an IBD patient with 3 or more of these miRNAs down-regulated is suspected of or identified as having dysplasia or cancer.

In another embodiment, the at least one or a plurality of miRNAs is selected from the group consisting of hsa-miR-30b, hsa-miR-106a, hsa-miR-15b, hsa-miR-649, hsa-miR-16, hsa-miR-652, hsa-let-7f, hsa-let-7g, hsa-miR-1287, hsa-miR-17, hsa-miR-1229, hsa-miR-28-3p, hsa-miR-182, hsa-let-7g*, and combinations thereof. In some instances, at least 1, 2, 3, 4, 5, 6, 7, 8, 9, 10, 11, 12, 13, or all 14 of these miRNAs are determined to establish a miRNA expression profile. In particular embodiments, an IBD patient with 3 or more of these miRNAs dysregulated (e.g., up-regulated and/or down-regulated) is suspected of or identified as having dysplasia or cancer. Table 1 illustrates the changes in the expression levels of these miRNAs when they are determined to be dysregulated in a patient sample (e.g., a sample from an IBD patient with dysplasia or cancer) as compared to the identical miRNA in a control and/or reference sample.

TABLE 1
Direction of change in the expression levels of
dysregulated miRNAs.

| | | | |
|---|---|---|---|
| hsa-miR-30b (“30b”) | ↓ | hsa-let-7g (“7g”) | ↓ |
| hsa-miR-106a (“106a”) | ↓ | hsa-miR-1287 (“1287”) | ↓ |
| hsa-miR-15b (“15b”) | ↓ | hsa-miR-17 (“17”) | ↑ |
| hsa-miR-649 (“649”) | ↑ | hsa-miR-1229 (“1229”) | ↓ |
| hsa-miR-16 (“16”) | ↓ | hsa-miR-28-3p (“28-3p”) | ↓ |
| hsa-miR-652 (“652”) | ↑ | hsa-miR-182 (“182”) | ↑ |
| hsa-let-7f (“7f”) | ↓ | hsa-let-7g* (“7g*”) | ↓ |

↑ up-regulated;
↓ down-regulated

In some embodiments, the methods of the present invention comprise determining the presence or level of at least 3 miRNAs to establish a miRNA expression profile, wherein the at least 3 miRNAs comprise 30b, 106a, and 15b; 30b, 106a, and 649; 30b, 106a, and 16; 30b, 106a, and 652; 30b, 106a, and 7f; 30b, 106a, and 7g; 30b, 106a, and 1287; 30b, 106a, and 17; 30b, 106a, and 1229; 30b, 106a, and 28-3p; 30b, 106a, and 182; 30b, 106a, and 7g*; 30b, 15b, and 649; 30b, 15b, and 16; 30b, 15b, and 652; 30b, 15b, and 7f; 30b, 15b, and 7g; 30b, 15b, and 1287; 30b, 15b, and 17; 30b, 15b, and 1229; 30b, 15b, and 28-3p; 30b, 15b, and 182; 30b, 15b, and 7g*; 30b, 649, and 16; 30b, 649, and 16; 30b, 649, and 652; 30b, 649, and 7f; 30b, 649, and 7g; 30b, 649, and 1287; 30b, 649, and 17; 30b, 649, and 1229; 30b, 649, and 28-3p; 30b, 649, and 182; 30b, 649, and 7g*; 30b, 16, and 652; 30b, 16, and 7f; 30b, 16, and 7g; 30b, 16, and 1287; 30b, 16, and 17; 30b, 16, and 1229; 30b, 16, and 28-3p; 30b, 16, and 182; 30b, 16, and 7g*; 30b, 652, and 7f; 30b, 652, and 7g; 30b, 652, and 1287; 30b, 652, and 17; 30b, 652, and 1229; 30b, 652, and 28-3p; 30b, 652, and 182; 30b, 652, and 7g*; 30b, 7f, and 7g; 30b, 7f, and 1287; 30b, 7f, and 17; 30b, 7f, and 1229; 30b, 7f, and 28-3p; 30b, 7f, and 182; 30b, 7f, and 7g*; 30b, 7g, and 1287; 30b, 7g, and 17; 30b, 7g, and 1229; 30b, 7g, and 28-3p; 30b, 7g, and 182; 30b, 7g, and 7g*; 30b, 1287, and 17; 30b, 1287, and 1229; 30b, 1287, and 28-3p; 30b, 1287, and 182; 30b, 1287, and 7g*; 30b, 17, and 1229; 30b, 17, and 28-3p; 30b, 17, and 182; 30b, 17, and 7g*; 30b, 1229, and 28-3p; 30b, 1229, and 182; 30b, 1229, and 7g*; 30b, 28-3p, and 182; 30b, 28-3p, and 7g*; or 30b, 182, and 7g*. The presence or level of at least 1, 2, 3, 4, 5, 6, 7, 8, 9, 10, 11, 12, 13, 14, 15, 20, 25, 30, 35, 40, 45, 50, or more additional miRNAs may be determined to establish the miRNA expression profile. In particular embodiments, an IBD patient with at least 1, 2, or all 3 of these miRNAs dysregulated (e.g., up-regulated and/or down-regulated as exemplified in Table 1) is suspected of or identified as having dysplasia or cancer.

In some embodiments, the methods of the present invention comprise determining the presence or level of at least 4 miRNAs to establish a miRNA expression profile, wherein the at least 4 miRNAs comprise 30b, 106a, 15b, and 649; 30b, 106a, 15b, and 16; 30b, 106a, 15b, and 652; 30b, 106a, 15b, and 7f; 30b, 106a, 15b, and 7g; 30b, 106a, 15b, and 1287; 30b, 106a, 15b, and 17; 30b, 106a, 15b, and 1229; 30b, 106a, 15b, and 28-3p; 30b, 106a, 15b, and 182; 30b, 106a, 15b, and 7g*; 30b, 15b, 649, and 16; 30b, 15b, 649, and 16; 30b, 15b, 649, and 652; 30b, 15b, 649, and 7f; 30b, 15b, 649, and 7g; 30b, 15b, 649, and 1287; 30b, 15b, 649, and 17; 30b, 15b, 649, and 1229; 30b, 15b, 649, and 28-3p; 30b, 15b, 649, and 182; 30b, 15b, 649, and 7g*; 30b, 649, 16, and 652; 30b, 649, 16, and 7f; 30b, 649, 16, and 7g; 30b, 649, 16, and 1287; 30b, 649, 16, and 17; 30b, 649, 16, and 1229; 30b, 649, 16, and 28-3p; 30b, 649, 16, and 182; 30b, 649, 16, and 7g*; 30b, 16, 652, and 7f; 30b, 16, 652, and 7g; 30b, 16, 652, and 1287; 30b, 16, 652, and 17; 30b, 16, 652, and 1229; 30b, 16, 652, and 28-3p; 30b, 16, 652, and 182; 30b, 16, 652, and 7g*; 30b, 652, 7f, and 7g; 30b, 652, 7f, and 1287; 30b, 652, 7f, and 17; 30b, 652, 7f, and 1229; 30b, 652, 7f, and 28-3p; 30b, 652, 7f, and 182; 30b, 652, 7f, and 7g*; 30b, 7f, 7g, and 1287; 30b, 7f, 7g, and 17; 30b, 7f, 7g, and 1229; 30b, 7f, 7g, and 28-3p; 30b, 7f, 7g, and 182; 30b, 7f, 7g, and 7g*; 30b, 7g, 1287, and 17; 30b, 7g, 1287, and 1229; 30b, 7g, 1287, and 28-3p; 30b, 7g, 1287, and 182; 30b, 7g, 1287, and 7g*; 30b, 1287, 17, and 1229; 30b, 1287, 17, and 28-3p; 30b, 1287, 17, and 182; 30b, 1287, 17, and 7g*; 30b, 17, 1229, and 28-3p; 30b, 17, 1229, and 28-3p; 30b, 17, 1229, and 182; 30b, 17, 1229, and 7g*; 30b, 1229, 28-3p, and 182; 30b, 1229, 28-3p, and 7g*; or 30b, 28-3p, 182, and 7g*. The presence or level of at least 1, 2, 3, 4, 5, 6, 7, 8, 9, 10, 11, 12, 13, 14, 15, 20, 25, 30, 35, 40, 45, 50, or more additional miRNAs may be determined to establish the miRNA expression profile. In particular embodiments, an IBD patient with at least 1, 2, 3, or all 4 of these miRNAs dysregulated (e.g., up-regulated and/or down-regulated as exemplified in Table 1) is suspected of or identified as having dysplasia or cancer.

In some embodiments, the methods of the present invention comprise determining the presence or level of at least 5 miRNAs to establish a miRNA expression profile, wherein the at least 5 miRNAs comprise 30b, 106a, 15b, 649, and 16; 30b, 106a, 15b, 649, and 652; 30b, 106a, 15b, 649, and 7f; 30b, 106a, 15b, 649, and 7g; 30b, 106a, 15b, 649, and 1287; 30b, 106a, 15b, 649, and 17; 30b, 106a, 15b, 649, and 1229; 30b, 106a, 15b, 649, and 28-3p; 30b, 106a, 15b, 649, and 182; 30b, 106a, 15b, 649, and 7g*; 30b, 15b, 649, 16, and 652; 30b, 15b, 649, 16, and 7f; 30b, 15b, 649, 16, and 7g; 30b, 15b, 649, 16, and 1287; 30b, 15b, 649, 16, and 17; 30b, 15b, 649, 16, and 1229; 30b, 15b, 649, 16, and 28-3p; 30b, 15b, 649, 16, and 182; 30b, 15b, 649, 16, and 7g*; 30b, 649, 16, 652, and 7f; 30b, 649, 16, 652, and 7g; 30b, 649, 16, 652, and 1287; 30b, 649, 16, 652, and 17; 30b, 649, 16, 652, and 1229; 30b, 649, 16, 652, and 28-3p; 30b, 649, 16, 652, and 182; 30b, 649, 16, 652, and 7g*; 30b, 16, 652, 7f, and 7g; 30b, 16, 652, 7f, and 1287; 30b, 16, 652, 7f, and 17; 30b, 16, 652, 7f, and 1229; 30b, 16, 652, 7f, and 28-3p; 30b, 16, 652, 7f, and 182; 30b, 16, 652, 7f, and 7g*; 30b, 652, 7f, 7g, and 1287; 30b, 652, 7f, 7g, and 17; 30b, 652, 7f, 7g, and 1229; 30b, 652, 7f, 7g, and 28-3p; 30b, 652, 7f, 7g, and 182; 30b, 652, 7f, 7g, and 7g*; 30b, 7f, 7g, 1287, and 17; 30b, 7f, 7g, 1287, and 1229; 30b, 7f, 7g, 1287, and 28-3p; 30b, 7f, 7g, 1287, and 182; 30b, 7f, 7g, 1287, and 7g*; 30b, 7g, 1287, 17, and 1229; 30b, 7g, 1287, 17, and 28-3p; 30b, 7g, 1287, 17, and 182; 30b, 7g, 1287, 17, and 7g*; 30b, 1287, 17, 1229, and 28-3p; 30b, 1287, 17, 1229, and 182; 30b, 1287, 17, 1229, and 7g*; 30b, 17, 1229, 28-3p, and 182; 30b, 17, 1229, 28-3p, and 7g*; or 30b, 1229, 28-3p, 182, and 7g*. The presence or level of at least 1, 2, 3, 4, 5, 6, 7, 8, 9, 10, 11, 12, 13, 14, 15, 20, 25, 30, 35, 40, 45, 50, or more additional miRNAs may be determined to establish the miRNA expression profile. In particular embodiments, an IBD patient with at least 1, 2, 3, 4, or all 5 of these miRNAs dysregulated (e.g., up-regulated and/or down-regulated as exemplified in Table 1) is suspected of or identified as having dysplasia or cancer.

In some embodiments, the methods of the present invention comprise determining the presence or level of at least 6 miRNAs to establish a miRNA expression profile, wherein the at least 6 miRNAs comprise 30b, 106a, 15b, 649, 16, and 652; 30b, 106a, 15b, 649, 16, and 7f; 30b, 106a, 15b, 649, 16, and 7g; 30b, 106a, 15b, 649, 16, and 1287; 30b, 106a, 15b, 649, 16, and 17; 30b, 106a, 15b, 649, 16, and 1229; 30b, 106a, 15b, 649, 16, and 28-3p; 30b, 106a, 15b, 649, 16, and 182; 30b, 106a, 15b, 649, 16, and 7g*; 30b, 15b, 649, 16, 652, and 7f; 30b, 15b, 649, 16, 652, and 7g; 30b, 15b, 649, 16, 652, and 1287; 30b, 15b, 649, 16, 652, and 17; 30b, 15b, 649, 16, 652, and 1229; 30b, 15b, 649, 16, 652, and 28-3p; 30b, 15b, 649, 16, 652, and 182; 30b, 15b, 649, 16, 652, and 7g*; 30b, 649, 16, 652, 7f, and 7g; 30b, 649, 16, 652, 7f, and 1287; 30b, 649, 16, 652, 7f, and 17; 30b, 649, 16, 652, 7f, and 1229; 30b, 649, 16, 652, 7f, and 28-3p; 30b, 649, 16, 652, 7f, and 182; 30b, 649, 16, 652, 7f, and 7g*; 30b, 16, 652, 7f, 7g, and 1287; 30b, 16, 652, 7f, 7g, and 17; 30b, 16, 652, 7f, 7g, and 1229; 30b, 16, 652, 7f, 7g, and 28-3p; 30b, 16, 652, 7f, 7g, and 182; 30b, 16, 652, 7f, 7g, and 7g*; 30b, 652, 7f, 7g, 1287, and 17; 30b, 652, 7f, 7g, 1287, and 1229; 30b, 652, 7f, 7g, 1287, and 28-3p; 30b, 652, 7f, 7g, 1287, and 182; 30b, 652, 7f, 7g, 1287, and 7g*; 30b, 7f, 7g, 1287, 17, and 1229; 30b, 7f, 7g, 1287, 17, and 28-3p; 30b, 7f, 7g, 1287, 17, and 182; 30b, 7f, 7g, 1287, 17, and 7g*; 30b, 7g, 1287, 17, 1229, and 28-3p; 30b, 7g, 1287, 17, 1229, and 182; 30b, 7g, 1287, 17, 1229, and 7g*; 30b, 1287, 17, 1229, 28-3p, and 182; 30b, 1287, 17, 1229, 28-3p, and 7g*; or 30b, 17, 1229, 28-3p, 182, and 7g*. The presence or level of at least 1, 2, 3, 4, 5, 6, 7, 8, 9, 10, 11, 12, 13, 14, 15, 20, 25, 30, 35, 40, 45, 50, or more additional miRNAs may be determined to establish the miRNA expression profile. In particular embodiments, an IBD patient with at least 1, 2, 3, 4, 5, or all 6 of these miRNAs dysregulated (e.g., up-regulated and/or down-regulated as exemplified in Table 1) is suspected of or identified as having dysplasia or cancer.

In some embodiments, the methods of the present invention comprise determining the presence or level of at least 7 miRNAs to establish a miRNA expression profile, wherein the at least 7 miRNAs comprise 30b, 106a, 15b, 649, 16, 652, and 7f; 30b, 106a, 15b, 649, 16, 652, and 7g; 30b, 106a, 15b, 649, 16, 652, and 1287; 30b, 106a, 15b, 649, 16, 652, and 17; 30b, 106a, 15b, 649, 16, 652, and 1229; 30b, 106a, 15b, 649, 16, 652, and 28-3p; 30b, 106a, 15b, 649, 16, 652, and 182; 30b, 106a, 15b, 649, 16, 652, and 7g*; 30b, 15b, 649, 16, 652, 7f, and 7g; 30b, 15b, 649, 16, 652, 7f, and 1287; 30b, 15b, 649, 16, 652, 7f, and 17; 30b, 15b, 649, 16, 652, 7f, and 1229; 30b, 15b, 649, 16, 652, 7f, and 28-3p; 30b, 15b, 649, 16, 652, 7f, and 182; 30b, 15b, 649, 16, 652, 7f, and 7g*; 30b, 649, 16, 652, 7f, 7g, and 1287; 30b, 649, 16, 652, 7f, 7g, and 17; 30b, 649, 16, 652, 7f, 7g, and 1229; 30b, 649, 16, 652, 7f, 7g, and 28-3p; 30b, 649, 16, 652, 7f, 7g, and 182; 30b, 649, 16, 652, 7f, 7g, and 7g*; 30b, 16, 652, 7f, 7g, 1287, and 17; 30b, 16, 652, 7f, 7g, 1287, and 1229; 30b, 16, 652, 7f, 7g, 1287, and 28-3p; 30b, 16, 652, 7f, 7g, 1287, and 182; 30b, 16, 652, 7f, 7g, 1287, and 7g*; 30b, 652, 7f, 7g, 1287, 17, and 1229; 30b, 652, 7f, 7g, 1287, 17, and 28-3p; 30b, 652, 7f, 7g, 1287, 17, and 182; 30b, 652, 7f, 7g, 1287, 17, and 7g*; 30b, 7f, 7g, 1287, 17, 1229, and 28-3p; 30b, 7f, 7g, 1287, 17, 1229, and 182; 30b, 7f, 7g, 1287, 17, 1229, and 7g*; 30b, 7g, 1287, 17, 1229, 28-3p, and 182; 30b, 7g, 1287, 17, 1229, 28-3p, and 7g*; or 30b, 1287, 17, 1229, 28-3p, 182, and 7g*. The presence or level of at least 1, 2, 3, 4, 5, 6, 7, 8, 9, 10, 11, 12, 13, 14, 15, 20, 25, 30, 35, 40, 45, 50, or more additional miRNAs may be determined to establish the miRNA expression profile. In particular embodiments, an IBD patient with at least 1, 2, 3, 4, 5, 6, or all 7 of these miRNAs dysregulated (e.g., up-regulated and/or down-regulated as exemplified in Table 1) is suspected of or identified as having dysplasia or cancer.

In some embodiments, the methods of the present invention comprise determining the presence or level of at least 8 miRNAs to establish a miRNA expression profile, wherein the at least 8 miRNAs comprise 30b, 106a, 15b, 649, 16, 652, 7f, and 7g; 30b, 106a, 15b, 649, 16, 652, 7f, and 1287; 30b, 106a, 15b, 649, 16, 652, 7f, and 17; 30b, 106a, 15b, 649, 16, 652, 7f, and 1229; 30b, 106a, 15b, 649, 16, 652, 7f, and 28-3p; 30b, 106a, 15b, 649, 16, 652, 7f, and 182; 30b, 106a, 15b, 649, 16, 652, 7f, and 7g*; 30b, 15b, 649, 16, 652, 7f, 7g, and 1287; 30b, 15b, 649, 16, 652, 7f, 7g, and 17; 30b, 15b, 649, 16, 652, 7f, 7g, and 1229; 30b, 15b, 649, 16, 652, 7f, 7g, and 28-3p; 30b, 15b, 649, 16, 652, 7f, 7g, and 182; 30b, 15b, 649, 16, 652, 7f, 7g, and 7g*; 30b, 649, 16, 652, 7f, 7g, 1287, and 17; 30b, 649, 16, 652, 7f, 7g, 1287, and 1229; 30b, 649, 16, 652, 7f, 7g, 1287, and 28-3p; 30b, 649, 16, 652, 7f, 7g, 1287, and 182; 30b, 649, 16, 652, 7f, 7g, 1287, and 7g*; 30b, 16, 652, 7f, 7g, 1287, 17, and 1229; 30b, 16, 652, 7f, 7g, 1287, 17, and 28-3p; 30b, 16, 652, 7f, 7g, 1287, 17, and 182; 30b, 16, 652, 7f, 7g, 1287, 17, and 7g*; 30b, 652, 7f, 7g, 1287, 17, 1229, and 28-3p; 30b, 652, 7f, 7g, 1287, 17, 1229, and 182; 30b, 652, 7f, 7g, 1287, 17, 1229, and 7g*; 30b, 7f, 7g, 1287, 17, 1229, 28-3p, and 182; 30b, 7f, 7g, 1287, 17, 1229, 28-3p, and 7g*; or 30b, 7g, 1287, 17, 1229, 28-3p, 182, and 7g*. The presence or level of at least 1, 2, 3, 4, 5, 6, 7, 8, 9, 10, 11, 12, 13, 14, 15, 20, 25, 30, 35, 40, 45, 50, or more additional miRNAs may be determined to establish the miRNA expression profile. In particular embodiments, an IBD patient with at least 1, 2, 3, 4, 5, 6, 7, or all 8 of these miRNAs dysregulated (e.g., up-regulated and/or down-regulated as exemplified in Table 1) is suspected of or identified as having dysplasia or cancer.

In some embodiments, the methods of the present invention comprise determining the presence or level of at least 9 miRNAs to establish a miRNA expression profile, wherein the at least 9 miRNAs comprise 30b, 106a, 15b, 649, 16, 652, 7f, 7g, and 1287; 30b, 106a, 15b, 649, 16, 652, 7f, 7g, and 17; 30b, 106a, 15b, 649, 16, 652, 7f, 7g, and 1229; 30b, 106a, 15b, 649, 16, 652, 7f, 7g, and 28-3p; 30b, 106a, 15b, 649, 16, 652, 7f, 7g, and 182; 30b, 106a, 15b, 649, 16, 652, 7f, 7g, and 7g*; 30b, 15b, 649, 16, 652, 7f, 7g, 1287, and 17; 30b, 15b, 649, 16, 652, 7f, 7g, 1287, and 1229; 30b, 15b, 649, 16, 652, 7f, 7g, 1287, and 28-3p; 30b, 15b, 649, 16, 652, 7f, 7g, 1287, and 182; 30b, 15b, 649, 16, 652, 7f, 7g, 1287, and 7g*; 30b, 649, 16, 652, 7f, 7g, 1287, 17, and 1229; 30b, 649, 16, 652, 7f, 7g, 1287, 17, and 28-3p; 30b, 649, 16, 652, 7f, 7g, 1287, 17, and 182; 30b, 649, 16, 652, 7f, 7g, 1287, 17, and 7g*; 30b, 16, 652, 7f, 7g, 1287, 17, 1229, and 28-3p; 30b, 16, 652, 7f, 7g, 1287, 17, 1229, and 182; 30b, 16, 652, 7f, 7g, 1287, 17, 1229, and 7g*; 30b, 652, 7f, 7g, 1287, 17, 1229, 28-3p, and 182; 30b, 652, 7f, 7g, 1287, 17, 1229, 28-3p, and 7g*; or 30b, 7f, 7g, 1287, 17, 1229, 28-3p, 182, and 7g*. The presence or level of at least 1, 2, 3, 4, 5, 6, 7, 8, 9, 10, 11, 12, 13, 14, 15, 20, 25, 30, 35, 40, 45, 50, or more additional miRNAs may be determined to establish the miRNA expression profile. In particular embodiments, an IBD patient with at least 1, 2, 3, 4, 5, 6, 7, 8, or all 9 of these miRNAs dysregulated (e.g., up-regulated and/or down-regulated as exemplified in Table 1) is suspected of or identified as having dysplasia or cancer.

In some embodiments, the methods of the present invention comprise determining the presence or level of at least 10 miRNAs to establish a miRNA expression profile, wherein the at least 10 miRNAs comprise 30b, 106a, 15b, 649, 16, 652, 7f, 7g, 1287, and 17; 30b, 106a, 15b, 649, 16, 652, 7f, 7g, 1287, and 1229; 30b, 106a, 15b, 649, 16, 652, 7f, 7g, 1287, and 28-3p; 30b, 106a, 15b, 649, 16, 652, 7f, 7g, 1287, and 182; 30b, 106a, 15b, 649, 16, 652, 7f, 7g, 1287, and 7g*; 30b, 15b, 649, 16, 652, 7f, 7g, 1287, 17, and 1229; 30b, 15b, 649, 16, 652, 7f, 7g, 1287, 17, and 28-3p; 30b, 15b, 649, 16, 652, 7f, 7g, 1287, 17, and 182; 30b, 15b, 649, 16, 652, 7f, 7g, 1287, 17, and 7g*; 30b, 649, 16, 652, 7f, 7g, 1287, 17, 1229, and 28-3p; 30b, 649, 16, 652, 7f, 7g, 1287, 17, 1229, and 182; 30b, 649, 16, 652, 7f, 7g, 1287, 17, 1229, and 7g*; 30b, 16, 652, 7f, 7g, 1287, 17, 1229, 28-3p, and 182; 30b, 16, 652, 7f, 7g, 1287, 17, 1229, 28-3p, and 7g*; or 30b, 652, 7f, 7g, 1287, 17, 1229, 28-3p, 182, and 7g*. The presence or level of at least 1, 2, 3, 4, 5, 6, 7, 8, 9, 10, 11, 12, 13, 14, 15, 20, 25, 30, 35, 40, 45, 50, or more additional miRNAs may be determined to establish the miRNA expression profile. In particular embodiments, an IBD patient with at least 1, 2, 3, 4, 5, 6, 7, 8, 9, or all 10 of these miRNAs dysregulated (e.g., up-regulated and/or down-regulated as exemplified in Table 1) is suspected of or identified as having dysplasia or cancer.

In some embodiments, the methods of the present invention comprise determining the presence or level of at least 11 miRNAs to establish a miRNA expression profile, wherein the at least 11 miRNAs comprise 30b, 106a, 15b, 649, 16, 652, 7f, 7g, 1287, 17, and 1229; 30b, 106a, 15b, 649, 16, 652, 7f, 7g, 1287, 17, and 28-3p; 30b, 106a, 15b, 649, 16, 652, 7f, 7g, 1287, 17, and 182; 30b, 106a, 15b, 649, 16, 652, 7f, 7g, 1287, 17, and 7g*; 30b, 15b, 649, 16, 652, 7f, 7g, 1287, 17, 1229, and 28-3p; 30b, 15b, 649, 16, 652, 7f, 7g, 1287, 17, 1229, and 182; 30b, 15b, 649, 16, 652, 7f, 7g, 1287, 17, 1229, and 7g*; 30b, 649, 16, 652, 7f, 7g, 1287, 17, 1229, 28-3p, and 182; 30b, 649, 16, 652, 7f, 7g, 1287, 17, 1229, 28-3p, and 7g*; or 30b, 16, 652, 7f, 7g, 1287, 17, 1229, 28-3p, 182, and 7g*. The presence or level of at least 1, 2, 3, 4, 5, 6, 7, 8, 9, 10, 11, 12, 13, 14, 15, 20, 25, 30, 35, 40, 45, 50, or more additional miRNAs may be determined to establish the miRNA expression profile. In particular embodiments, an IBD patient with at least 1, 2, 3, 4, 5, 6, 7, 8, 9, 10, or all 11 of these miRNAs dysregulated (e.g., up-regulated and/or down-regulated as exemplified in Table 1) is suspected of or identified as having dysplasia or cancer.

In some embodiments, the methods of the present invention comprise determining the presence or level of at least 12 miRNAs to establish a miRNA expression profile, wherein the at least 12 miRNAs comprise 30b, 106a, 15b, 649, 16, 652, 7f, 7g, 1287, 17, 1229, and 28-3p; 30b, 106a, 15b, 649, 16, 652, 7f, 7g, 1287, 17, 1229, and 182; 30b, 106a, 15b, 649, 16, 652, 7f, 7g, 1287, 17, 1229, and 7g*; 30b, 15b, 649, 16, 652, 7f, 7g, 1287, 17, 1229, 28-3p, and 182; 30b, 15b, 649, 16, 652, 7f, 7g, 1287, 17, 1229, 28-3p, and 7g*; or 30b, 649, 16, 652, 7f, 7g, 1287, 17, 1229, 28-3p, 182, and 7g*. The presence or level of at least 1, 2, 3, 4, 5, 6, 7, 8, 9, 10, 11, 12, 13, 14, 15, 20, 25, 30, 35, 40, 45, 50, or more additional miRNAs may be determined to establish the miRNA expression profile. In particular embodiments, an IBD patient with at least 1, 2, 3, 4, 5, 6, 7, 8, 9, 10, 11, or all 12 of these miRNAs dysregulated (e.g., up-regulated and/or down-regulated as exemplified in Table 1) is suspected of or identified as having dysplasia or cancer.

In some embodiments, the methods of the present invention comprise determining the presence or level of at least 13 miRNAs to establish a miRNA expression profile, wherein the at least 13 miRNAs comprise 30b, 106a, 15b, 649, 16, 652, 7f, 7g, 1287, 17, 1229, 28-3p, and 182; 30b, 106a, 15b, 649, 16, 652, 7f, 7g, 1287, 17, 1229, 28-3p, and 7g*; or 30b, 15b, 649, 16, 652, 7f, 7g, 1287, 17, 1229, 28-3p, 182, and 7g*. The presence or level of at least 1, 2, 3, 4, 5, 6, 7, 8, 9, 10, 11, 12, 13, 14, 15, 20, 25, 30, 35, 40, 45, 50, or more additional miRNAs may be determined to establish the miRNA expression profile. In particular embodiments, an IBD patient with at least 1, 2, 3, 4, 5, 6, 7, 8, 9, 10, 11, 12, or all 13 of these miRNAs dysregulated (e.g., up-regulated and/or down-regulated as exemplified in Table 1) is suspected of or identified as having dysplasia or cancer.

In some embodiments, the methods of the present invention comprise determining the presence or level of at least 14 miRNAs to establish a miRNA expression profile, wherein the at least 14 miRNAs comprise 30b, 106a, 15b, 649, 16, 652, 7f, 7g, 1287, 17, 1229, 28-3p, 182, and 7g*. The presence or level of at least 1, 2, 3, 4, 5, 6, 7, 8, 9, 10, 11, 12, 13, 14, 15, 20, 25, 30, 35, 40, 45, 50, or more additional miRNAs may be determined to establish the miRNA expression profile. In particular embodiments, an IBD patient with at least 1, 2, 3, 4, 5, 6, 7, 8, 9, 10, 11, 12, 13, or all 14 of these miRNAs dysregulated (e.g., up-regulated and/or down-regulated as exemplified in Table 1) is suspected of or identified as having dysplasia or cancer.

In some embodiments, the sample is whole blood, serum, or plasma. In particular embodiments, the IBD patient is a human. In certain instances, the IBD patient is a Crohn's disease (CD) patient (i.e., has CD). In other instances, the IBD patient is an ulcerative colitis (UC) patient (i.e., has UC).

In certain embodiments, the dysplasia is selected from the group consisting of flat dysplasia, elevated dysplasia (e.g., polypoid dysplasia, dysplasia associated lesion or mass (DALM)), and indefinite dysplasia. Examples of flat dysplasia include, without limitation, flat low-grade dysplasia and flat high-grade dysplasia. Non-limiting examples of elevated dysplasia include adenoma-like DALM and non-adenoma-like DALM.

In some instances, the cancer is selected from the group consisting of small intestine (e.g., small bowel) cancer, colorectal cancer, gastrointestinal stromal tumors, gastrointestinal carcinoid tumors, colon cancer, rectal cancer, anal cancer, bile duct cancer, stomach cancer, esophageal cancer, gallbladder cancer, liver cancer, pancreatic cancer, appendix cancer, lung cancer, breast cancer, ovarian cancer, renal cancer, cancer of the central nervous system, skin cancer, lymphomas, brain cancer, prostate cancer, ovarian cancer, uterine cancer, head and neck cancers, and combinations thereof. In particular embodiments, the cancer is selected from the group consisting of small bowel cancer, colorectal cancer, and combinations thereof.

In certain instances, the IBD patient is identified as having or suspected of having CD with small bowel cancer. The methods of the invention are particularly advantageous for identifying CD patients with small bowel cancer due to the inaccessibility of this cancer type to endoscopy. In certain other instances, the IBD patient is identified as having or suspected of having CD with dysplasia (e.g., flat dysplasia, elevated dysplasia, indefinite dysplasia).

In some instances, the IBD patient is identified as having or suspected of having UC with colorectal cancer. In other instances, the IBD patient is identified as having or suspected of having UC with dysplasia (e.g., flat dysplasia, elevated dysplasia, indefinite dysplasia).

The methods of the present invention are also useful for predicting whether an IBD patient will develop dysplasia or cancer. In certain instances, the methods described herein can be used to predict the likelihood or probability of an IBD patient developing dysplasia or cancer sometime in the future (e.g., days, months, or years after initial IBD diagnosis).

In certain embodiments, the methods of the present invention may further comprise recommending and/or performing a colonoscopy when an IBD patient is identified as having or suspected of having or likely to develop dysplasia or cancer.

In certain embodiments, the methods of the present invention may further comprise recommending and/or administering one or more anticancer drugs (e.g., chemotherapy) when an IBD patient is identified as having or suspected of having or likely to develop dysplasia or cancer. Suitable non-limiting examples of anticancer drugs are described herein.

In certain embodiments, the methods of the present invention may further comprise recommending and/or performing surgery (e.g., colectomy and/or polypectomy) when an IBD patient is identified as having or suspected of having or likely to develop dysplasia or cancer.

In certain embodiments, the methods of the present invention are also useful for predicting, identifying, and/or determining whether a non-IBD patient has, is suspected of having, or will develop sporadic colorectal cancer.

In certain embodiments, the dysplastic or cancer sample profile is compared to the model using a statistical algorithm. Preferably, the statistical algorithm comprises a binary decision tree, a k-Nearest Neighbor (kNN) prediction algorithm, or a combination thereof.

In other aspects, the present invention provides a method for monitoring the efficacy of treatment of an IBD patient with dysplasia or cancer, the method comprising:
(a) determining (e.g., measuring) the presence or level (e.g., expression level) of at least one or a plurality of (e.g., at least 1, 2, 3, 4, 5, 6, 7, 8, 9, 10, 11, 12, 13, 14, 15, or more) miRNAs in a first sample (e.g., a blood sample) from the patient to establish a first miRNA expression profile, wherein the first sample is obtained before treatment;
(b) determining (e.g., measuring) the presence or level (e.g., expression level) of the same miRNAs in a second sample (e.g., a blood sample) from the patient to establish a second miRNA expression profile, wherein the second sample is obtained during or after treatment;
(c) comparing the first miRNA expression profile to the second miRNA expression profile; and
(d) monitoring the efficacy of treatment for the IBD patient (e.g., whether the patient is in remission) based upon the comparison in step (c).

In certain embodiments, the method further comprises comparing the first miRNA expression profile with one or more pre-established model miRNA expression profiles from a control and/or reference standard (e.g., an IBD control sample without dysplasia or cancer, a non-IBD cancer sample such as a non-IBD colorectal cancer sample, etc.).

In other embodiments, the method further comprises comparing the second miRNA expression profile with one or more pre-established model miRNA expression profiles from a control and/or reference standard (e.g., an IBD control sample without dysplasia or cancer, a non-IBD cancer sample such as a non-IBD colorectal cancer sample, etc.).

In some embodiments, the efficacy of treatment for an IBD patient is monitored by determining whether at least one or a plurality of (e.g., at least 2, 3, 4, 5, 6, 7, 8, 9, 10, 11, 12, 13, 14, 15, or more) of the miRNAs in the first and/or second miRNA expression profile(s) is dysregulated (e.g., up-regulated and/or down-regulated) compared to the identical miRNAs in one or more pre-established model miRNA expression profiles. In particular embodiments, a miRNA in an IBD patient is dysregulated when there is at least a 1.5, 2, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 15, 20, 25, 30, 35, 40, 45, 50, or more fold change in expression (e.g., up-regulation or down-regulation) compared to the identical miRNA in a control and/or reference sample. In preferred embodiments, a miRNA in the first and/or second miRNA expression profile is dysregulated when there is at least about a 2-fold change in its expression level compared to the identical miRNA in the one or more pre-established model miRNA expression profiles.

In certain embodiments, the at least one or a plurality of miRNAs is selected from the group consisting of hsa-miR-16, hsa-miR-15b, hsa-miR-15a, hsa-let-7f, hsa-miR-30b, hsa-miR-649, hsa-miR-575, hsa-miR-106a, hsa-let-7g, hsa-miR-223, hsa-miR-17, hsa-miR-652, hsa-miR-1287, hsa-miR-1229, hsa-miR-21, hsa-miR-28-3p, hsa-miR-182, hsa-let-7g*, and combinations thereof. In some instances, at least 1, 2, 3, 4, 5, 6, 7, 8, 9, 10, 11, 12, 13, 14, 15, 16, 17, or 18 of these miRNAs (alone or in combination with at least one or a plurality of other miRNAs) are determined to establish both the first and second miRNA expression profiles. In some embodiments, the IBD patient is identified as being responsive to treatment (e.g., treatment is effective and/or patient is in remission) when at least 1, 2, 3, 4, 5, 6, 7, 8, 9, 10, 11, 12, 13, 14, 15, 16, 17, or 18 of these miRNAs are determined to be dysregulated (e.g., up-regulated and/or down-regulated) in the first miRNA expression profile but the identical miRNAs are determined not to be dysregulated (e.g., have normal expression) in the second miRNA expression profile. In yet other embodiments, the IBD patient is identified as being unresponsive to treatment (e.g., treatment is not effective and/or the patient still has IBD) when at least 1, 2, 3, 4, 5, 6, 7, 8, 9, 10, 11, 12, 13, 14, 15, 16, 17, or 18 of these miRNAs are determined to be dysregulated (e.g., up-regulated and/or down-regulated) in the first miRNA expression profile and the identical miRNAs are also dysregulated in the second miRNA expression profile.

In one embodiment, the at least one or a plurality of miRNAs is selected from the group consisting of hsa-miR-16, hsa-miR-15b, hsa-miR-15a, hsa-let-7f, hsa-miR-30b, hsa-miR-649, hsa-miR-575, hsa-miR-106a, hsa-let-7g, hsa-miR-223, hsa-miR-17, hsa-miR-652, hsa-miR-1287, hsa-miR-1229, hsa-miR-21, and combinations thereof. In some instances, at least 1, 2, 3, 4, 5, 6, 7, 8, 9, 10, 11, 12, 13, 14, or all 15 of these miRNAs are determined to establish both the first and second miRNA expression profiles. In particular embodiments, the IBD patient is identified as being responsive to treatment (e.g., treatment is effective and/or patient is in remission) when 3 or more of these miRNAs are down-regulated in the first miRNA expression profile but the identical miRNAs are not dysregulated (e.g., have normal expression) in the second miRNA expression profile. In other embodiments, the IBD patient is identified as being unresponsive to treatment (e.g., treatment is not effective and/or the patient still has IBD) when 3 or more of these miRNAs are down-regulated in the first miRNA expression profile and the identical miRNAs are also down-regulated in the second miRNA expression profile.

In another embodiment, the at least one or a plurality of miRNAs is selected from the group consisting of hsa-miR-30b, hsa-miR-106a, hsa-miR-15b, hsa-miR-649, hsa-miR-16, hsa-miR-652, hsa-let-7f, hsa-let-7g, hsa-miR-1287, hsa-miR-17, hsa-miR-1229, hsa-miR-28-3p, hsa-miR-182, hsa-let-7g*, and combinations thereof. In some instances, at least 1, 2, 3, 4, 5, 6, 7, 8, 9, 10, 11, 12, 13, or all 14 of these miRNAs are determined to establish both the first and second miRNA expression profiles. In particular embodiments, the IBD patient is identified as being responsive to treatment (e.g., treatment is effective and/or patient is in remission) when 3 or more of these miRNAs are dysregulated (e.g., up-regulated and/or down-regulated) in the first miRNA expression profile but the identical miRNAs are not dysregulated (e.g., have normal expression) in the second miRNA expression profile. In other embodiments, the IBD patient is identified as being unresponsive to treatment (e.g., treatment is not effective and/or the patient still has IBD) when 3 or more of these miRNAs are dysregulated (e.g., up-regulated and/or down-regulated) in the first miRNA expression profile and the identical miRNAs are also dysregulated in the second miRNA expression profile. Table 1 illustrates the changes in the expression levels of these miRNAs when they are determined to be dysregulated in a patient sample (e.g., a sample from an IBD patient with dysplasia or cancer) as compared to the identical miRNA in a control and/or reference sample.

In some embodiments, the methods of the present invention comprise determining the presence or level of any of the above-described combinations of at least 3, 4, 5, 6, 7, 8, 9, 10, 11, 12, 13, or 14 of the following miRNAs to establish a first miRNA expression profile and/or a second miRNA expression profile: 30b, 106a, 15b, 649, 16, 652, 7f, 7g, 1287, 17, 1229, 28-3p, 182, and 7g*. The presence or level of at least 1, 2, 3, 4, 5, 6, 7, 8, 9, 10, 11, 12, 13, 14, 15, 20, 25, 30, 35, 40, 45, 50, or more additional miRNAs may also be determined to establish the first and/or second miRNA expression profile. In particular embodiments, an IBD patient with at least 1, 2, 3, 4, 5, 6, 7, 8, 9, 10, 11, 12, 13, or all 14 of these miRNAs dysregulated (e.g., up-regulated and/or down-regulated as exemplified in Table 1) in the first miRNA expression profile but the identical miRNAs are not dysregulated (e.g., have normal expression) in the second miRNA expression profile is identified as responsive to treatment (e.g., treatment is effective and/or patient is in remission). In other particular embodiments, an IBD patient with at least 1, 2, 3, 4, 5, 6, 7, 8, 9, 10, 11, 12, 13, or all 14 of these miRNAs dysregulated (e.g., up-regulated and/or down-regulated as exemplified in Table 1) in the first miRNA expression profile and the identical miRNAs are also dysregulated in the second miRNA expression profile is identified as unresponsive to treatment (e.g., treatment is not effective and/or the patient still has IBD).

In some embodiments, the treatment for the IBD patient comprises therapy with one or more anticancer drugs (e.g., chemotherapy), surgery, or combinations thereof.

In certain embodiments, the anticancer drug comprises an anti-signaling agent (i.e., a cytostatic drug) such as a monoclonal antibody or a tyrosine kinase inhibitor; an anti-proliferative agent; a chemotherapeutic agent (i.e., a cytotoxic drug); a hormonal therapeutic agent; a radiotherapeutic agent; a vaccine; and/or any other compound with the ability to reduce or abrogate the uncontrolled growth of aberrant cells such as dysplastic or cancerous cells. In some embodiments, the isolated cells are treated with one or more anti-signaling agents, anti-proliferative agents, and/or hormonal therapeutic agents in combination with at least one chemotherapeutic agent.

Non-limiting examples of anti-signaling agents include monoclonal antibodies such as trastuzumab (Herceptin®), pertuzumab (2C4), alemtuzumab (Campath®), bevacizumab (Avastin®), cetuximab (Erbitux®), gemtuzamab (Mylotarg®), panitumumab (Vectibix™) rituximab (Rituxan®), and tositumomab (BEXXAR®); tyrosine kinase inhibitors such as gefitinib (Iressa®), sunitinib (Sutent®), erlotinib (Tarceva®), lapatinib (GW-572016; Tykerb®), canertinib (CI-1033), semaxinib (SU5416), vatalanib (PTK787/ZK222584), sorafenib (BAY 43-9006; Nexavar®), imatinib mesylate (Gleevec®), leflunomide (SU101), vandetanib (ZACTIMA™; ZD6474), pelitinib, CP-654577, CP-724714, HKI-272, PKI-166, AEE788, BMS-599626, HKI-357, BIBW 2992, ARRY-334543, JNJ-26483327, and JNJ-26483327; and combinations thereof.

Exemplary anti-proliferative agents include mTOR inhibitors such as sirolimus (rapamycin), temsirolimus (CCI-779), everolimus (RAD001), BEZ235, and XL765; AKT inhibitors such as 1L6-hydroxymethyl-chiro-inositol-2-(R)-2-O-methyl-3-O-octadecyl-sn-glycerocarbonate, 9-methoxy-2-methylellipticinium acetate, 1,3-dihydro-1-((4(4-(6-phenyl-1H-imidazo[4,5-g]quinoxalin-7-yl)phenyl)methyl)-4-piperidinyl)-2H-benzimidazol-2-one, 10-(4′-(N-diethylamino)butyl)-2-chlorophenoxazine, 3-formylchromone thiosemicarbazone (Cu(II)Cl₂ complex), API-2, a 15-mer peptide derived from amino acids 10-24 of the proto-oncogene TCL1 (Hiromura et al., J. Biol. Chem., 279:53407-53418 (2004), KP372-1, and the compounds described in Kozikowski et al., J. Am. Chem. Soc., 125:1144-1145 (2003) and Kau et al., Cancer Cell, 4:463-476 (2003); PI3K inhibitors such as PX-866, wortmannin, LY 294002, quercetin, tetrodotoxin citrate, thioperamide maleate, GDC-0941 (957054-30-7), IC87114, PI-103, PIK93, BEZ235 (NVP-BEZ235), TGX-115, ZSTK474, (-)-deguelin, NU 7026, myricetin, tandutinib, GDC-0941 bismesylate, GSK690693, KU-55933, MK-2206, OSU-03012, perifosine, triciribine, XL-147, PIK75, TGX-221, NU 7441, PI 828, XL-765, and WHI-P 154; MEK inhibitors such as PD98059, ARRY-162, RDEA119, U0126, GDC-0973, PD184161, AZD6244, AZD8330, PD0325901, and ARRY-142886; and combinations thereof.

Non-limiting examples of chemotherapeutic agents include platinum-based drugs (e.g., oxaliplatin, cisplatin, carboplatin, spiroplatin, iproplatin, satraplatin, etc.), alkylating agents (e.g., cyclophosphamide, ifosfamide, chlorambucil, busulfan, melphalan, mechlorethamine, uramustine, thiotepa, nitrosoureas, etc.), anti-metabolites (e.g., 5-fluorouracil, azathioprine, 6-mercaptopurine, methotrexate, leucovorin, capecitabine, cytarabine, floxuridine, fludarabine, gemcitabine (Gemzar®), pemetrexed (ALIMTA®), raltitrexed, etc.), plant alkaloids (e.g., vincristine, vinblastine, vinorelbine, vindesine, podophyllotoxin, paclitaxel (Taxon®), docetaxel (Taxotere®), etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), pharmaceutically acceptable salts thereof, stereoisomers thereof, derivatives thereof, analogs thereof, and combinations thereof.

Examples of hormonal therapeutic agents include, without limitation, aromatase inhibitors (e.g., aminoglutethimide, anastrozole (Arimidex®), letrozole (Femara®), vorozole, exemestane (Aromasin®), 4-androstene-3,6,17-trione (6-OXO), 1,4,6-androstatrien-3,17-dione (ATD), formestane (Lentaron®), etc.), selective estrogen receptor modulators (e.g., bazedoxifene, clomifene, fulvestrant, lasofoxifene, raloxifene, tamoxifen, toremifene, etc.), steroids (e.g., dexamethasone), finasteride, and gonadotropin-releasing hormone agonists (GnRH) such as goserelin, pharmaceutically acceptable salts thereof, stereoisomers thereof, derivatives thereof, analogs thereof, and combinations thereof.

Non-limiting examples of cancer vaccines useful in the present invention include ANYARA from Active Biotech, DCVax-LB from Northwest Biotherapeutics, EP-2101 from IDM Pharma, GV1001 from Pharmexa, IO-2055 from Idera Pharmaceuticals, INGN 225 from Introgen Therapeutics and Stimuvax from Biomira/Merck.

Examples of radiotherapeutic agents include, but are not limited to, radionuclides such as ⁴⁷Sc, ⁶⁴Cu, ⁶⁷Cu, ⁸⁹Sr, ⁸⁶Y, ⁸⁷Y, ⁹⁰Y, ¹⁰⁵Rh, ¹¹¹Ag, ¹¹¹In, ^{117m}Sn, ¹⁴⁹Pm, ¹⁵³Sm, ¹⁶⁶Ho, ¹⁷⁷Lu, ¹⁸⁶Re, ¹⁸⁸Re, ²¹¹At, and ²¹²Bi, optionally conjugated to antibodies directed against tumor antigens.

Non-limiting examples of surgical procedures suitable for treating IBD patients with dysplasia or cancer include colectomy, polypectomy, and combinations thereof.

In some embodiments, the first and second samples are independently selected from the group consisting of whole blood, serum, and plasma. In preferred embodiments, the first and second samples are the same type of sample, e.g., whole blood. In other embodiments, the IBD patient is a human. In certain instances, the IBD patient is a Crohn's disease (CD) patient (i.e., has CD). In other instances, the IBD patient is an ulcerative colitis (UC) patient (i.e., has UC).

In certain embodiments, the dysplasia is selected from the group consisting of flat dysplasia, elevated dysplasia (e.g., polypoid dysplasia, dysplasia associated lesion or mass (DALM)), and indefinite dysplasia. Examples of flat dysplasia include, without limitation, flat low-grade dysplasia and flat high-grade dysplasia. Non-limiting examples of elevated dysplasia include adenoma-like DALM and non-adenoma-like DALM.

In some instances, the cancer is selected from the group consisting of small intestine (e.g., small bowel) cancer, colorectal cancer, gastrointestinal stromal tumors, gastrointestinal carcinoid tumors, colon cancer, rectal cancer, anal cancer, bile duct cancer, stomach cancer, esophageal cancer, gallbladder cancer, liver cancer, pancreatic cancer, appendix cancer, lung cancer, breast cancer, ovarian cancer, renal cancer, cancer of the central nervous system, skin cancer, lymphomas, brain cancer, prostate cancer, ovarian cancer, uterine cancer, head and neck cancers, and combinations thereof. In particular embodiments, the cancer is selected from the group consisting of small bowel cancer, colorectal cancer, and combinations thereof.

In certain instances, the IBD patient has CD with small bowel cancer. In other instances, the IBD patient has CD with dysplasia (e.g., flat dysplasia, elevated dysplasia, indefinite dysplasia).

In certain instances, the IBD patient has UC with colorectal cancer. In further instances, the IBD patient has UC with dysplasia (e.g., flat dysplasia, elevated dysplasia, indefinite dysplasia).

In certain embodiments, the methods of the present invention may further comprise maintaining the current course of therapy by recommending and/or administering the same anticancer drug(s) (e.g., chemotherapy), e.g., at the same dose with the same dosing regimen, when the IBD patient is identified as being responsive to treatment.

In other embodiments, the methods of the present invention may further comprise adjusting the current course of therapy by recommending and/or administering the same anticancer drug(s) (e.g., chemotherapy) at a different dose and/or with a different dosing regimen, recommending and/or administering one or more different anticancer drugs (e.g., chemotherapy), and/or recommending and/or performing surgery (e.g., colectomy and/or polypectomy) when the IBD patient is identified as being unresponsive to treatment.

In certain embodiments, the methods of the present invention are also useful for monitoring the efficacy of treatment for a non-IBD patient with sporadic colorectal cancer.

In certain other embodiments, the first and second miRNA expression profiles are compared to each other and/or independently compared to one or more pre-established model profiles using a statistical algorithm. Preferably, the statistical algorithm comprises a binary decision tree, a k-Nearest Neighbor (kNN) prediction algorithm, or a combination thereof.

### IV. miRNA Extraction, Purification, and Enrichment

Once a sample such as blood has been isolated or obtained from a patient, small RNA species such as miRNAs may be extracted, purified, and/or enriched from the sample (e.g., from a cellular extract or lysate thereof) by any technique known in the art. In some embodiments, commercially available miRNA extraction kits (e.g., PAXgene miRNA kit, Qiagen; mirVana miRNa isolation kit, Ambion; and mirPremier microRNA isolation kit, Sigma-Aldrich) can be used to isolate miRNAs.

In some instances, an alcohol solution may be added to, mixed with, or incubated with the sample prior to extraction of miRNAs. The alcohol solution may comprise at least one alcohol and typically ranges from about 5% to about 100% in the concentration of the alcohol. In exemplary embodiments, the amount of alcohol solution added to the sample renders it with an alcohol concentration of about 35% to about 70%, or about 50% to about 60%. In other exemplary embodiments, the amount of alcohol solution added to the sample gives it an alcohol concentration of about 55%. Suitable alcohols include, but are not limited to, ethanol, propanol, isopropanol, methanol, and mixtures thereof. It is further contemplated that an alcohol solution may be used in additional steps in methods for precipitating RNA.

In certain aspects, miRNAs may be extracted from the sample (e.g., from a cellular extract or lysate thereof) with an extraction solution comprising a non-alcohol organic solvent prior to applying the sample to a solid support. In exemplary embodiments, the extraction solution contains a non-alcohol organic solvent such as phenol and/or chloroform. The non-alcohol organic solvent solution is understood to contain at least one non-alcohol organic solvent, though it may also contain an alcohol. The concentrations described above with respect to alcohol solutions are applicable to concentrations of solutions having non-alcohol organic solvents. In certain embodiments, equal amounts of the sample and phenol and/or chloroform are mixed. In specific embodiments, the alcohol solution is added to the sample before extraction with a non-alcohol organic solvent.

In some embodiments, extraction of miRNAs from the sample (e.g., from a cellular extract or lysate thereof) includes using a solid support, such as a mineral or polymer support. A “solid support” includes, e.g., a physical structure containing a material which contacts the sample and that does not irreversibly react to macromolecules in the lysate, particularly with small RNA molecules such as miRNAs. In particular embodiments, the solid support binds small RNA molecules; in additional cases, it binds small RNA molecules, but does not bind one or more other types of macromolecules in the sample. The material in the solid support may include a mineral or polymer, in which case the support is referred to as a “mineral or polymer support.” Mineral or polymer supports include supports involving silica. In some embodiments, the silica is glass. Suitable supports include, but are not limited to, beads, columns, and filters. In further embodiments, the mineral or polymer support is a glass fiber filter (GFF) or column.

In certain other embodiments, the mineral or polymer support may include polymers or nonpolymers with electronegative groups. In some embodiments, the material comprises polyacrylate, polystyrene, latex, polyacrylonitrile, polyvinylchloride, methacrylate, and/or methyl methacrylate.

In further embodiments, a sample that may or may not have been mixed with an alcohol or non-alcohol organic solvent solution is applied to a solid support and the RNA (containing miRNAs) is eluted from the support.

After a sample (e.g., a cellular extract or lysate thereof) is applied or mixed with a solid support, the material may be washed with a solution. In some embodiments, a mineral or polymer support is washed with a first wash solution after applying the sample to the mineral or polymer support. In further embodiments, a wash solution comprises a chaotropic or reducing agent. The chaotropic agent is guanidinium in some wash solutions. A wash solution includes alcohol in some embodiments, and in some cases, it has both alcohol and guanidinium. It is further contemplated that the extraction step may include 1, 2, 3, 4, 5, or more washes with a wash solution. The wash solution used when more than one washing is involved may be the same or different. In some embodiments, the wash solutions have the same components, but in different concentrations from each other. It is generally understood that molecules that come through the material in a wash cycle are discarded.

The desired RNA molecules are typically eluted from the solid support. In certain embodiments, small RNA molecules (e.g., miRNAs) are eluted from a solid support such as a mineral or polymer support at a temperature of about 60° C. to about 100° C. The temperature at which the RNA molecules are eluted may be about or at least about 5° C. to about 100° C. or more, or any range therein. The molecules may be eluted with any elution solution. In some embodiments, the elution solution is an ionic solution. In particular embodiments, the elution solution includes up to about 10 mM salt (e.g., about 0.1, 0.5, 1, 5, 10, or more mM salt). In certain embodiments, the salt consists of a combination of Li⁺, Na⁺, K⁺, or NH₄⁺ as the cation and Cl⁻, Br⁻, I⁻, ethylenediaminetetraacetate, or citrate as the anion.

Additional steps include passing the small RNA molecules through a glass fiber filter (GFF) while binding only the larger RNAs. In some embodiments, the passed small RNA molecules are captured on a second GFF and then eluted. Material that is not captured on the second GFF filter may be discarded or not used.

In a specific embodiment, the extraction of miRNAs is performed as follows: adding an extraction solution to a sample (e.g., a cellular extract or lysate thereof) containing miRNAs; adding an alcohol solution to the extracted sample; applying the sample to a mineral or polymer support; and eluting the RNA containing miRNAs from the mineral or polymer support with an ionic solution. In some embodiments, the eluted sample is enriched at least about 10-fold for miRNAs by mass.

As a non-limiting example, the extraction, purification, and enrichment of miRNAs from a blood sample may be performed according to the following protocol. 60 μl of 2M Na-acetate, pH 4.0, is added to the sample (e.g., a cellular extract or lysate thereof), followed immediately by 0.6 ml of acid phenol-chloroform. In certain instances, ethanol is added to the sample before phenol-chloroform extraction to provide a final concentration of about 55% ethanol. After 30 sec of vigorous agitation, the aqueous phase is separated by centrifugation at 16,000×G for 5 min. Four 100 μl aliquots of this aqueous phase are used in four separate separations. The four aliquots have 100 μl of 40%, 50%, 60%, and 70% ethanol added to each, and then are passed through glass fiber filters as in the RNAqueous procedure (Ambion, Inc.; Austin, Tex.). The 20%, 25%, 30%, and 35% ethanol solutions that passed through these filters (the flow-through) are then adjusted to 55% ethanol final concentration by the addition of 156, 133, 111, and 88.9 μl of ethanol, respectively. All four samples are passed over separate glass fiber filter columns. The filters are then washed with 0.7 ml of 4 M guanidinium isocyanate (GuSCN)/70% ethanol, followed by two washes with 0.5 ml 80% alcohol/0.1 M NaCl/4.5 mM EDTA/10 mM TrisHCl, pH 7.5. After each wash is passed through the filter, the collection tube is emptied and replaced. Each wash is passed through the filter by centrifugation as per the RNAqueous protocol (Ambion, Inc.). The sample is then eluted off the filter with 100 μl of 0.1 mM EDTA, pH 8.0, which is applied directly to the filter at room temperature and centrifuged through into a fresh collection tube.

Additional methods for extracting, purifying, and enriching miRNAs are described in, e.g., U.S. Patent Publication No. 20050059024; and the mirVana™ miRNA Isolation Kit Protocol (Ambion, Inc.; Austin, Tex.), the disclosures of which are herein incorporated by reference in their entirety for all purposes.

### V. Exemplary miRNA Sequences

The present invention provides, inter alia, assay methods for identifying miRNA expression patterns from blood obtained from a subject. In certain embodiments, the patient will have IBD with or without dysplasia or cancer. In particular embodiments, the miRNA expression profile indicates that the patient has dysplasia or cancer, e.g., identifies an IBD patient as having dysplasia or cancer.

As described herein, the determination of a specific miRNA expression profile in a blood sample advantageously enables the identification of the presence of dysplasia or cancer in the subject.

As used herein, “microRNA,” “miRNA,” or “miR” includes the unprocessed (e.g., precursor) or processed (e.g., mature) RNA transcript from a miR gene. The unprocessed miR gene transcript is also called a “miR precursor” or “miR prec” and typically comprises an RNA transcript of about 70-100 nucleotides in length. The miR precursor can be processed by digestion with an RNAse (e.g., Dicer, Argonaut, or RNAse III such as E. coli RNAse III) into an active 19-25 nucleotide RNA molecule. This active 19-25 nucleotide RNA molecule is also called the “processed” miR gene transcript or “mature” miRNA. In certain embodiments, the “mature” miRNA of 19-25 nucleotides is the preferred miRNA of the present invention.

Table 2 below depicts the nucleotide sequences of particular precursor and mature human miRNAs.

Additional precursor and mature miRNA sequences may be found in the miRBase Sequence Database, available at http://microrna.sanger.ac.uk/sequences/.

Further information regarding any of the 677 miRNAs identified to date in humans is available at http://www.microrna.org/.

TABLE 2
Exemplary Precursor miRNA and Mature miRNA.

| miRNA | Precursor Sequence | Mature Sequence |
|---|---|---|
| hsa-let-7f-1 | UCAGAGUGAGGUAGUAGAUUGUAUAGUUGUGGGG | UGAGGUAGUAGAUUGUAUAGUU |
| | UAGUGAUUUUACCCUGUUCAGGAGAUAACUAUACA | |
| | AUCUAUUGCCUUCCCUGA | |
| hsa-let-7f-2 | UGUGGGAUGAGGUAGUAGAUUGUAUAGUUUUAGG | UGAGGUAGUAGAUUGUAUAGUU |
| | GUCAUACCCCAUCUUGGAGAUAACUAUACAGUCUA | |
| | CUGUCUUUCCCACG | |
| hsa-let-7g | AGGCUGAGGUAGUAGUUUGUACAGUUUGAGGGUCU | UGAGGUAGUAGUUUGUACAGUU |
| | AUGAUACCACCCGGUACAGGAGAUAACUGUACAGG | |
| | CCACUGCCUUGCCA | |
| hsa-let-7g* | AGGCUGAGGUAGUAGUUUGUACAGUUUGAGGGUCU | CUGUACAGGCCACUGCCUUGC |
| | AUGAUACCACCCGGUACAGGAGAUAACUGUACAGG | |
| | CCACUGCCUUGCCA | |
| hsa-miR-15a | CCUUGGAGUAAAGUAGCAGCACAUAAUGGUUUGUG | UAGCAGCACAUAAUGGUUUGUG |
| | GAUUUUGAAAAGGUGCAGGCCAUAUUGUGCUGCCU | |
| | CAAAAAUACAAGG | |
| hsa-miR-15b | UUGAGGCCUUAAAGUACUGUAGCAGCACAUCAUGG | UAGCAGCACAUCAUGGUUUACA |
| | UUUACAUGCUACAGUCAAGAUGCGAAUCAUUAUUU | |
| | GCUGCUCUAGAAAUUUAAGGAAAUUCAU | |
| hsa-miR-16-1 | GUCAGCAGUGCCUUAGCAGCACGUAAAUAUUGGCG | UAGCAGCACGUAAAUAUUGGCG |
| | UUAAGAUUCUAAAAUUAUCUCCAGUAUUAACUGUG | |
| | CUGCUGAAGUAAGGUUGAC | |
| hsa-miR-16-2 | GUUCCACUCUAGCAGCACGUAAAUAUUGGCGUAGU | UAGCAGCACGUAAAUAUUGGCG |
| | GAAAUAUAUAUUAAACACCAAUAUUACUGUGCUGC | |
| | UUUAGUGUGAC | |
| hsa-miR-17 | GUCAGAAUAAUGUCAAAGUGCUUACAGUGCAGGUA | CAAAGUGCUUACAGUGCAGGUAG |
| | GUGAUAUGUGCAUCUACUGCAGUGAAGGCACUUGU | |
| | AGCAUUAUGGUGAC | |
| hsa-miR-21 | UGUCGGGUAGCUUAUCAGACUGAUGUUGACUGUUG | CAACACCAGUCGAUGGGCUGU |
| | AAUCUCAUGGCAACACCAGUCGAUGGGCUGUCUGA | |
| | CA | |
| hsa-miR-28-3p | GGUCCUUGCCCUCAAGGAGCUCACAGUCUAUUGAG | CACUAGAUUGUGAGCUCCUGGA |
| | UUACCUUUCUGACUUUCCCACUAGAUUGUGAGCUC | |
| | CUGGAGGGCAGGCACU | |
| hsa-miR-30b | ACCAAGUUUCAGUUCAUGUAAACAUCCUACACUCA | UGUAAACAUCCUACACUCAGCU |
| | GCUGUAAUACAUGGAUUGGCUGGGAGGUGGAUGUU | |
| | UACUUCAGCUGACUUGGA | |
| hsa-miR-106a | CCUUGGCCAUGUAAAAGUGCUUACAGUGCAGGUAG | AAAAGUGCUUACAGUGCAGGUAG |
| | CUUUUUGAGAUCUACUGCAAUGUAAGCACUUCUUA | |
| | CAUUACCAUGG | |
| hsa-miR-182 | GAGCUGCUUGCCUCCCCCCGUUUUUGGCAAUGGUA | UUUGGCAAUGGUAGAACUCACACU |
| | GAACUCACACUGGUGAGGUAACAGGAUCCGGUGGU | |
| | UCUAGACUUGCCAACUAUGGGGCGAGGACUCAGCC | |
| | GGCAC | |
| hsa-miR-223 | CCUGGCCUCCUGCAGUGCCACGCUCCGUGUAUUUG | CGUGUAUUUGACAAGCUGAGUU |
| | ACAAGCUGAGUUGGACACUCCAUGUGGUAGAGUGU | |
| | CAGUUUGUCAAAUACCCCAAGUGCGGCACAUGCUU | |
| | ACCAG | |
| hsa-miR-575 | AAUUCAGCCCUGCCACUGGCUUAUGUCAUGACCUU | GAGCCAGUUGGACAGGAGC |
| | GGGCUACUCAGGCUGUCUGCACAAUGAGCCAGUUG | |
| | GACAGGAGCAGUGCCACUCAACUC | |
| hsa-miR-649 | GGCCUAGCCAAAUACUGUAUUUUUGAUCGACAUUU | AAACCUGUGUUGUUCAAGAGUC |
| | GGUUGAAAAAUAUCUAUGUAUUAGUAAACCUGUGU | |
| | UGUUCAAGAGUCCACUGUGUUUUGCUG | |
| hsa-miR-652 | ACGAAUGGCUAUGCACUGCACAACCCUAGGAGAGG | AAUGGCGCCACUAGGGUUGUG |
| | GUGCCAUUCACAUAGACUAUAAUUGAAUGGCGCCA | |
| | CUAGGGUUGUGCAGUGCACAACCUACAC | |
| hsa-miR-1229 | GUGGGUAGGGUUUGGGGGAGAGCGUGGGCUGGGGU | CUCUCACCACUGCCCUCCCACAG |
| | UCAGGGACACCCUCUCACCACUGCCCUCCCACAG | |
| hsa-miR-1287 | GUUGUGCUGUCCAGGUGCUGGAUCAGUGGUUCGAG | UGCUGGAUCAGUGGUUCGAGUC |
| | UCUGAGCCUUUAAAAGCCACUCUAGCCACAGAUGC | |
| | AGUGAUUGGAGCCAUGACAA | |

### VI. Measuring miRNA Expression

The presence or level of at least one miRNA in blood or circulating cells such as circulating tumor cells (CTCs) can be measured using a variety of techniques that are well known to those of skill in the art (e.g., quantitative or semi-quantitative RT-PCR, microarray analysis, Northern blot analysis, solution hybridization detection, and the like). Additional techniques suitable for measuring miRNA expression are described in, e.g., U.S. Patent Publication No. 2008/0306006, the disclosure of which is herein incorporated by reference in its entirety for all purposes.

In some embodiments, the presence or level of at least one miRNA is measured by reverse transcribing RNA from a test sample (e.g., blood sample) obtained from a subject to provide a set of target oligodeoxynucleotides, hybridizing the target oligodeoxynucleotides to one or more miRNA-specific probe oligonucleotides (e.g., hybridizing to a microarray that comprises several miRNA-specific probe oligonucleotides) to provide a hybridization profile for the test sample, and comparing the test sample hybridization profile to a hybridization profile from a control sample or reference standard. In certain embodiments, an alteration in the signal of at least one miRNA in the test sample from a subject relative to the control sample is indicative of dysplasia or cancer (e.g., blood cells) and the identity of the presence of dysplasia or cancer in the subject. In one particular embodiment, target oligonucleotides are hybridized to a microarray comprising miRNA-specific probe oligonucleotides for one or more miRNAs selected from the group consisting of hsa-miR-16, hsa-miR-15b, hsa-miR-15a, hsa-let-7f, hsa-miR-30b, hsa-miR-649, hsa-miR-575, hsa-miR-106a, hsa-let-7g, hsa-miR-223, hsa-miR-17, hsa-miR-652, hsa-miR-1287, hsa-miR-1229, hsa-miR-21, hsa-miR-28-3p, hsa-miR-182, hsa-let-7g*, and combinations thereof. In one embodiment, the microarray comprises miRNA-specific probe oligonucleotides for a substantial portion of all known human miRNAs.

In other embodiments, the present invention provides multiplex detection of target nucleic acids in a sample. As used herein, the phrase “multiplex” includes the detection of more than one target nucleic acid of interest within a single reaction. In one embodiment of the invention, multiplex includes the detection of between 2-10,000 different target nucleic acids in a single reaction. As used herein, multiplex includes the detection of any range between 2-10,000, e.g., between 2-100 different target nucleic acids in a single reaction, 5-500 different target nucleic acids in a single reaction, 25-1000 different target nucleic acids, 10-100 different target nucleic acids in a single reaction, for example, 5, 10, 15, 20, 25, 30, 35, 40, 45, 50, 55, 60, such as 2, 3, 4, 5, 6, 7, 8, 9, 10, 11, 12, 13, 14, 15, 16, 17, 18, 19, 20, 21, 22, 23, 24, 25 26, 27, 28, 29, 30, 31, 21, 33, 34, 35, 36, 37, 38, 39, and 40. Preferably, the nucleic acid is a miRNA.

The present invention also provides high throughput detection and analysis of target nucleic acids in a sample. As used herein, the phrase “high throughput” includes the detection or analysis of more than one reaction in a single process, where each reaction is itself a multiplex reaction, detecting more than one target nucleic acid of interest. In one preferred embodiment, 2-10,000 multiplex reactions can be processed simultaneously.

An “expression profile” or “hybridization profile” of a particular sample such as a whole blood or CTC sample is essentially a fingerprint of the state of the sample. While two states can have any particular miRNA similarly or differentially expressed, the evaluation of a number of miRNAs simultaneously allows the generation of a miRNA expression profile that is unique to the state of the sample. That is, normal tissue can be distinguished from dysplastic and/or cancerous (e.g., tumor) tissue, and within dysplastic or cancerous tissue, different prognosis states (e.g., good or poor long-term survival prospects) can be determined. By comparing the expression profiles of blood or circulating cells such as CTCs in different states and/or to normal tissue, information regarding which miRNAs are important (including both up- and down-regulation of miRNAs) in each of these states or relative to normal tissue is obtained. The identification of sequences that are differentially expressed in blood as well as the differential expression resulting in different prognostic outcomes, allows the use of this information in a number of ways. For example, a particular dosing or treatment regimen can be evaluated (e.g., to determine whether a chemotherapeutic drug acts to improve the long-term prognosis in a particular patient) and/or implemented. Similarly, diagnosis and/or prognosis can be performed or confirmed by comparing patient samples with known miRNA expression profiles. Furthermore, these miRNA expression profiles (or individual miRNAs) allow the screening of drug candidates that suppress the miRNA expression profile or convert a poor prognosis profile to a better prognosis profile.

The microarray described herein can be prepared from gene-specific oligonucleotide probes generated from known miRNA sequences. The array may contain two different oligonucleotide probes for each miRNA, one containing the active, mature sequence and the other being specific for the precursor of the miRNA. The array may also contain controls, such as one or more mouse sequences differing from human orthologs by only a few bases, which can serve as controls for hybridization stringency conditions. tRNAs or other RNAs (e.g., rRNAs, mRNAs, etc.) from both species may also be printed on the microchip, providing an internal, relatively stable, positive control for specific hybridization. One or more appropriate controls for non-specific hybridization may also be included on the microchip. For this purpose, sequences are selected based upon the absence of any homology with any known miRNAs.

In certain aspects, the methods of the present invention are useful for a variety of miRNA expression profiling applications. More particularly, the invention encompasses methods for high-throughput genetic screening. In some embodiments, the methods allow the rapid and simultaneous detection of multiple defined target nucleic acids such as mRNA or miRNA sequences in nucleic samples obtained from a multiplicity of individuals. It can be carried out by simultaneously amplifying many different target sequences from a large number of desired samples, such as patient nucleic acid samples, using the methods described above.

In general, as used herein, an expression signature is a set of miRNAs, where the expression level of an individual miRNA differs between a first physiological state or condition relative to its expression level in a second physiological state or condition, i.e., state A and state B. As one non-limiting example, an expression signature may comprise a set of miRNAs which differ in the level of expression between dysplastic cells and non-dysplastic cells, or between dysplastic cells of one type or stage and dysplastic cells of a different type or stage. As another non-limiting example, an expression signature may comprise a set of miRNAs which differ in the level of expression between cancerous cells and non-cancerous cells, or between cancerous cells from one tumor type or stage and cancerous cells from a different tumor type or stage.

The terms “differentially expressed miRNA,” “differential miRNA expression” and their synonyms include a miRNA whose expression is activated to a higher or lower level in one physiological state relative to a second physiological subject suffering from a disease, such as dysplasia and/or cancer, relative to its expression in a normal or control subject. A differentially expressed miRNA can be either activated or inhibited at the nucleic acid level or protein level, or may be subject to alternative splicing to result in a different polypeptide product. Such differences may be evidenced, e.g., by a change in mRNA levels or miRNA levels, surface expression, secretion or other partitioning of a polypeptide. Differential miRNA expression is considered to be present when there is at least about two-fold, at least about three-fold, at least about four-fold, at least about six-fold, or at least about ten-fold difference between the expression of a given miRNA between two different physiological states, such as in various stages of disease development in a diseased individual.

An expression signature is sometimes referred to herein as a set of miRNAs. In certain instances, an expression signature, or set of marker miRNAs, is a minimum number that is capable of identifying a phenotypic state of a cell. A set of marker miRNAs that is representative of a cellular phenotype is one which includes a minimum number of miRNAs that identify markers to demonstrate that a cell has a particular phenotype. In general, two discrete cell populations in different physiological states having the desired phenotypes can be examined by the methods of the present invention. The minimum number of miRNAs in a set of marker genes typically depends on the particular phenotype being examined. In some embodiments, the minimum number is 2, 3, 4, 5, or more miRNAs. In other embodiments, the minimum number is 5, 10, 15, 20, 25, 30, 35, 40, 45, 50, 55, 60, 65, 70, 75, 80, 85, 90, 95, 100, 500, 1000, or more miRNAs.

### VII. Analysis of miRNA Expression

In one aspect, the present invention provides methods for screening for the presence of malignant cells in a test sample by determining the level of expression of total miRNAs in a test sample; and comparing the levels of expression of miRNAs of the test sample and a control sample. The control sample can be either a normal non-malignant sample or a malignant sample. In one aspect, a lower level of expression of miRNAs in the test sample compared to the control sample is indicative of the test sample containing malignant cells. One can use any screening method known in the art, including, e.g., a solution based method or other known methods such as micorarrays for miRNAs, such as that described in Miska et al., Genome Biol., 5(9):R68 (2004).

Qualitative analyses can be performed comparing miRNA profiles in IBD patients with or without colorectal neoplasia. miRNA profiles in blood obtained prior to colonoscopy or surgery can be correlated with the presence or absence of colorectal neoplasia determined histologically. In certain instances, subjects will have a blood test prior to colonoscopy, colectomy, or surgery. In some instances, the invention will aid in the diagnosis of colorectal neoplasia in patients with IBD and potentially obviate the need for routine colonoscopy. Given the imperfect sensitivity of colonoscopy, a blood-based diagnostic test to identify dysplasia or cancer such as colorectal cancer would advance patient care.

In some embodiments, the present invention provides methods for screening an individual at risk for dysplasia or cancer by obtaining at least two samples (e.g., blood or cells isolated from blood) from the individual at different times; and comparing the level of expression of miRNAs in the samples, wherein a lower level of expression of miRNAs in the later obtained sample compared to the earlier obtained sample indicates that the individual is at risk for dysplasia or cancer.

In particular embodiments, the methods of the present invention are useful for characterizing poorly differentiated tumors. As exemplified herein, miRNA expression distinguishes tumors from normal tissues, even for poorly differentiated tumors. In certain instances, miRNAs are expressed at lower levels in tumors compared to normal tissues, irrespective of cell type.

In other embodiments, the present invention provides methods for determining the presence or level of miRNAs from whole blood or rare circulating cells such as CTCs that are particularly useful for detecting or identifying dysplasia or cancer. In certain instances, the expression profile of miRNAs in an IBD patient suspected of having or developing dysplasia or cancer can be compared to a set of miRNA expression profiles for an IBD patient without dysplasia or cancer, allowing classification of the test samples to be assessed based on the comparison.

In another embodiment, the level of expression for a specific group of miRNAs from whole blood, sometimes referred to as a profile group of miRNAs, is determined, where lower expression of the profile group of miRNAs is associated with risk for a particular type and/or stage of dysplasia or cancer.

In addition, in certain other aspects, the methods described herein can be used in conjunction with panels of gene expression markers that identify dysplastic or cancerous cells. As a non-limiting example, these gene panels can be useful in conjunction with the methods of the present invention in identifying individuals with a metastatic digestive and gastrointestinal cancer (e.g., metastatic colorectal cancer or small bowel cancer) who would benefit from therapy consistent with that given to those individuals diagnosed initially with such cancer. Suitable systems include, but are not limited to, the Rosetta Genomics CUP assay (see, e.g., PCT Publication No. WO 08/117,278); the Aviara DX (Carlsbad, Calif.) CancerTYPE ID™ assay, an RT-PCR-based expression assay that measures 92 genes to identify the primary site of origin for 39 tumor types; and the Pathwork™ Tissue of Origin Test (Sunnyvale, Calif.), which measures the expression of more than 1600 genes on a microarray and compares a tumor's gene expression “signature” against those of 15 known tissue types.

### VIII. IBD, Dysplasia, and Cancers

Inflammatory bowel disease (IBD) is a group of inflammatory conditions of the large intestine and small intestine. The main forms of IBD are Crohn's disease (CD) and ulcerative colitis (UC). Other less common forms of IBD include, e.g., indeterminate colitis (IC), collagenous colitis, lymphocytic colitis, ischemic colitis, diversion colitis, Behçet's syndrome, infective colitis, and the like.

Crohn's disease (CD) is a disease of chronic inflammation that can involve any part of the gastrointestinal tract. Commonly, the distal portion of the small intestine, i.e., the ileum, and the cecum are affected. In other cases, the disease is confined to the small intestine, colon, or anorectal region. CD occasionally involves the duodenum and stomach, and more rarely the esophagus and oral cavity.

A hallmark of CD is the presence of discrete aggregations of inflammatory cells, known as granulomas, which are generally found in the submucosa. Some CD cases display typical discrete granulomas, while others show a diffuse granulomatous reaction or a nonspecific transmural inflammation. As a result, the presence of discrete granulomas is indicative of CD, although the absence of granulomas is also consistent with the disease. Thus, transmural or discontinuous inflammation, rather than the presence of granulomas, is a preferred diagnostic indicator of CD (see, e.g., Rubin and Farber, Pathology (2nd Edition), Philadelphia, J.B. Lippincott Company (1994)).

Ulcerative colitis (UC) is a disease of the large intestine characterized by chronic diarrhea with cramping, abdominal pain, rectal bleeding, loose discharges of blood, pus, and mucus. The manifestations of UC vary widely. A pattern of exacerbations and remissions typifies the clinical course for about 70% of UC patients, although continuous symptoms without remission are present in some patients with UC. Local and systemic complications of UC include arthritis, eye inflammation such as uveitis, skin ulcers, and liver disease. In addition, UC, and especially the long-standing, extensive form of the disease is associated with an increased risk of colon carcinoma.

UC is a diffuse disease that usually extends from the most distal part of the rectum for a variable distance proximally. The term “left-sided colitis” describes an inflammation that involves the distal portion of the colon, extending as far as the splenic flexure. Sparing of the rectum or involvement of the right side (proximal portion) of the colon alone is unusual in UC. The inflammatory process of UC is limited to the colon and does not involve, for example, the small intestine, stomach, or esophagus. In addition, UC is distinguished by a superficial inflammation of the mucosa that generally spares the deeper layers of the bowel wall. Crypt abscesses, in which degenerated intestinal crypts are filled with neutrophils, are also typical of UC (see, e.g., Rubin and Farber, supra).

As described above, the term “cancer” is intended to include any member of a class of diseases characterized by the uncontrolled growth of aberrant cells. Preferably, the cancer is a digestive or gastrointestinal cancer including, but not limited to, colorectal cancer, small intestine (small bowel) cancer, gastrointestinal stromal tumors, gastrointestinal carcinoid tumors, colon cancer, rectal cancer, anal cancer, bile duct cancer, gastric (stomach) cancer, esophageal cancer, appendix cancer, and combinations thereof.

The term “colorectal cancer” is intended to include a cancer that starts in the large intestine (colon) or the rectum (end of the colon).

As described above, a dysplasia typically includes abnormal cell or tissue growth. Dysplasia is sometimes indicative of an early neoplastic or cancer process. The term includes a condition in which there is an expansion of immature cells and/or a delay in the maturation and differentiation of mature cells.

In certain embodiments, at least one miRNA in a group of miRNAs is used for identification of dysplasia or a stage or type thereof in a subject with IBD. The at least one miRNA in the group of miRNAs may be selected from the group consisting of hsa-miR-16, hsa-miR-15b, hsa-miR-15a, hsa-let-7f, hsa-miR-30b, hsa-miR-649, hsa-miR-575, hsa-miR-106a, hsa-let-7g, hsa-miR-223, hsa-miR-17, hsa-miR-652, hsa-miR-1287, hsa-miR-1229, hsa-miR-21, hsa-miR-28-3p, hsa-miR-182, hsa-let-7g*, and combinations thereof, including at least 1, 2, 3, 4, 5, 6, 7, 8, 9, 10, 11, 12, 13, 14, 15, 16, 17, or all 18 miRNAs.

In certain other embodiments, at least one miRNA in a group of miRNAs is used for identification of cancer or a stage or type thereof in a subject with IBD. The at least one miRNA in the group of miRNAs may be selected from the group consisting of hsa-miR-16, hsa-miR-15b, hsa-miR-15a, hsa-let-7f, hsa-miR-30b, hsa-miR-649, hsa-miR-575, hsa-miR-106a, hsa-let-7g, hsa-miR-223, hsa-miR-17, hsa-miR-652, hsa-miR-1287, hsa-miR-1229, hsa-miR-21, hsa-miR-28-3p, hsa-miR-182, hsa-let-7g*, and combinations thereof, including 1, 2, 3, 4, 5, 6, 7, 8, 9, 10, 11, 12, 13, 14, 15, 16, 17, or all 18 miRNAs.

In yet other embodiments, at least one miRNA in a group of miRNAs is used for identification of colorectal cancer or a stage or type thereof in a subject with IBD. The at least one miRNA in the group of miRNAs may be selected from the group consisting of hsa-miR-16, hsa-miR-15b, hsa-miR-15a, hsa-let-7f, hsa-miR-30b, hsa-miR-649, hsa-miR-575, hsa-miR-106a, hsa-let-7g, hsa-miR-223, hsa-miR-17, hsa-miR-652, hsa-miR-1287, hsa-miR-1229, hsa-miR-21, hsa-miR-28-3p, hsa-miR-182, hsa-let-7g*, and combinations thereof, including 1, 2, 3, 4, 5, 6, 7, 8, 9, 10, 11, 12, 13, 14, 15, 16, 17, or all 18 miRNAs.

### IX. miRNA Expression Profiles Classify Human Dysplasias and Cancers

Various statistical algorithms can be used in order to analyze the expression profiles of various miRNAs. In certain instances, the algorithms herein comprise one or more learning statistical classifier systems. The term “learning statistical classifier system” includes a machine learning algorithmic technique capable of adapting to complex data sets and making decisions based upon such data sets. In some embodiments, a single learning statistical classifier system such as a classification tree (e.g., random forest) is used. In other embodiments, a combination of 2 or more learning statistical classifier systems can be used, preferably in tandem.

Examples of learning statistical classifier systems include, but are not limited to, Hierarchical Clustering, binary decision trees, k-Nearest Neighbor (kNN) Prediction, Probabilistic Neural Network (PNN) Prediction, those using inductive learning (e.g., decision/classification trees such as random forests, classification and regression trees (C&RT), boosted trees, etc.), Probably Approximately Correct (PAC) learning, connectionist learning (e.g., neural networks (NN), artificial neural networks (ANN), neuro fuzzy networks (NFN), network structures, perceptrons such as multi-layer perceptrons, multi-layer feed-forward networks, applications of neural networks, Bayesian learning in belief networks, etc.), reinforcement learning (e.g., passive learning in a known environment such as naïve learning, adaptive dynamic learning, and temporal difference learning, passive learning in an unknown environment, active learning in an unknown environment, learning action-value functions, applications of reinforcement learning, etc.), and genetic algorithms and evolutionary programming. Other learning statistical classifier systems include support vector machines (e.g., Kernel methods), multivariate adaptive regression splines (MARS), Levenberg-Marquardt algorithms, Gauss-Newton algorithms, mixtures of Gaussians, gradient descent algorithms, and learning vector quantization (LVQ).

Random forests are learning statistical classifier systems that are constructed using an algorithm developed by Leo Breiman and Adele Cutler. Random forests use a large number of individual decision trees and decide the class by choosing the mode (i.e., most frequently occurring) of the classes as determined by the individual trees. Random forest analysis can be performed, e.g., using the RandomForests software available from Salford Systems (San Diego, Calif.). See, e.g., Breiman, Machine Learning, 45:5-32 (2001); and http://stat-www.berkeley.edu/users/breiman/RandomForests/cc_home.htm, for a description of random forests.

Classification and regression trees represent a computer intensive alternative to fitting classical regression models and are typically used to determine the best possible model for a categorical or continuous response of interest based upon one or more predictors. Classification and regression tree analysis can be performed, e.g., using the CART software available from Salford Systems or the Statistica data analysis software available from StatSoft, Inc. (Tulsa, Okla.). A description of classification and regression trees is found, e.g., in Breiman et al. “Classification and Regression Trees,” Chapman and Hall, New York (1984); and Steinberg et al., “CART: Tree-Structured Non-Parametric Data Analysis,” Salford Systems, San Diego, (1995).

Binary decision trees are essentially computer science binary tree structures, enabling a conclusion state to be reached from a root node (e.g., a question or decision choice) via a set of binary (e.g., yes/no) decision states. Binary decision trees are powerful tools for classification and prediction that allow a conclusion to be made based on a specified problem definition. A binary decision tree typically comprises of a set of body nodes which are attached to a root node and which terminate at n leaf nodes. The root and each body node have connections to two other nodes; otherwise, they are classed as terminating nodes where a decision outcome state has been reached. For example, the leaf nodes in a binary decision tree may represent a set of terminating “answers” or decision outcome states, and the root and body nodes may represent the “questions.” The binary decision tree arrives at a decision state by gaining answers to the body (e.g., yes/no) nodes. In certain instances, the nature of the response to a particular question dictates which node should be followed to the next question (or answer if a leaf node is arrived at). In the case of a binary decision tree, the response corresponds to one of the two available branches at each body node. Binary decision trees may be implemented in C++ or any other programming language known to one of skill in the art.

Neural networks are interconnected groups of artificial neurons that use a mathematical or computational model for information processing based on a connectionist approach to computation. Typically, neural networks are adaptive systems that change their structure based on external or internal information that flows through the network. Specific examples of neural networks include feed-forward neural networks such as perceptrons, single-layer perceptrons, multi-layer perceptrons, backpropagation networks, ADALINE networks, MADALINE networks, Learnmatrix networks, radial basis function (RBF) networks, and self-organizing maps or Kohonen self-organizing networks; recurrent neural networks such as simple recurrent networks and Hopfield networks; stochastic neural networks such as Boltzmann machines; modular neural networks such as committee of machines and associative neural networks; and other types of networks such as instantaneously trained neural networks, spiking neural networks, dynamic neural networks, and cascading neural networks. Neural network analysis can be performed, e.g., using the Statistica data analysis software available from StatSoft, Inc. See, e.g., Freeman et al., In “Neural Networks: Algorithms, Applications and Programming Techniques,” Addison-Wesley Publishing Company (1991); Zadeh, Information and Control, 8:338-353 (1965); Zadeh, “IEEE Trans. on Systems, Man and Cybernetics,” 3:28-44 (1973); Gersho et al., In “Vector Quantization and Signal Compression,” Kluywer Academic Publishers, Boston, Dordrecht, London (1992); and Hassoun, “Fundamentals of Artificial Neural Networks,” MIT Press, Cambridge, Mass., London (1995), for a description of neural networks.

In one specific example, a binary decision tree comprises a decision tree with multiple nodes and at each node the branches can go left or right (binary) regarding the analysis (e.g., comparison) of miRNAs at that specific node. There may be about 20-25 (e.g., 20, 21, 22, 23, 24, 25, or less or more) nodes in the tree, the same number of miRNAs (e.g., 1, 2, 3, or more at each node) and about 20-30 (e.g., 20, 21, 22, 23, 24, 25, 26, 27, 28, 29, 30, or less or more) different cancer types. The analysis starts at the top and traverses the tree, making decisions at each node to go left or right depending on the miRNA analysis (e.g., comparison) at that node.

In one non-limiting example, the structure of a binary decision tree classifier can have 4 nodes and 5 leaves. Each node is a binary decision between two sets of samples, those to the left and right of the node. A series of binary decisions, starting at node #1 and moving downwards, lead to one of the possible tumor types, which are the “leaves” of the tree. Decisions are made at consecutive nodes using miRNA expression levels, until an end-point (“leaf of the tree”) is reached, indicating the predicted class for this sample. For example, a sample which is classified as “lung” (i.e., lung cancer) must undergo the path through nodes #1, #2, and #4, taking the right branch at nodes #1 and #4 and the left branch at node #2, with no decision needed at node #3.

In another specific example, miRNA expression data first undergoes filtering. The purpose of this filtering is to remove miRNAs which have no detectable expression and thus are uninformative, but can introduce noise to the clustering. A miRNA is regarded as “not expressed” or “not detectable” if that particular miRNA does not have an expression value above a minimal cutoff in any of the samples. In one preferred embodiment, hierarchical clustering is used for this filtering step.

Next, after filtration, miRNA selection is performed on all the detectable miRNAs. Nominal P-values are calculated for each miRNA, kNN prediction is then performed using kNN software. kNN software is a predicting algorithm that learns from a training data set and predicts samples in a test data set. A set of miRNAs (e.g., miRNAs that best distinguish two classes of samples, in this case, normal vs. tumor) are selected using the training data set. Distances between the test samples and the training samples are measured in the space of the selected miRNAs. Prediction can be performed, one test sample at a time, by (i) identifying the k nearest samples (neighbors) of the test sample among the training data set; and (ii) assigning the test sample to the majority class of these k samples.

In certain preferred embodiments, the kNN algorithm complements and adds precision to the binary decision tree prediction regarding the analysis (e.g., comparison) of miRNAs. In one exemplary embodiment, the binary decision tree identifies the presence of dysplasia or cancer or a type or stage thereof for most patient samples, but the kNN algorithm may be used in conjunction to take the decision process to the next level for more difficult cases. In essence, the nearest neighboring points are also taken into consideration to refine the outcome, thereby enabling the identification and/or the classification of the dysplasia or cancer. A non-limiting example of a binary decision tree/kNN analysis is described in PCT Publication No. WO 08/117,278, the disclosure of which is herein incorporated by reference in its entirety for all purposes.

### X. Anticancer Therapies

As explained in greater detail herein, the results of the methods of the present invention allow for the identification of dysplasia or cancer. In certain instances, the whole blood or rare circulating cells can also be isolated from a patient sample during IBD drug treatment and stimulated with one or more growth factors to determine whether a specific therapy should be implemented. As such, the methods of the invention advantageously assist the clinician in providing the right anticancer drug at the right dose at the right time for every patient.

In contrast to currently available colorectal cancer testing options such as colonoscopy, the present invention enables the identification and/or monitoring of cancer patients with a non-invasive procedure. As such, the methods of the invention, which utilize the detection of one or a plurality of miRNAs in samples such as blood or circulating cells, provide valuable diagnostic and prognostic information for individualized cancer therapy (e.g., “personalized medicine”) that is not possible with tissue biopsy samples.

### XI. Examples

The following examples are offered to illustrate, but not to limit, the claimed invention.

### Example 1

### Clinical Study Protocol for Identifying Blood microRNA Expression Patterns in Inflammatory Bowel Disease (IBD) Patients with Dysplasia or Cancer

This example describes a pilot study in which microRNA profiles in serum obtained prior to colonoscopy or surgery are correlated with the presence or absence of colorectal neoplasia determined histologically. All subjects have a blood test prior to colonoscopy or surgery. IBD patients undergoing every 1-2 year surveillance colonoscopies are eligible for repeat blood draws on an every 1-2 year basis up to 3 years.

Patients with inflammatory bowel disease (IBD) have an increased risk of developing colorectal neoplasia including dysplasia and colorectal cancer (CRC). The standard of care in 2010 is to perform surveillance colonoscopies every 1-2 years with biopsies in patients with longstanding and extensive IBD to identify dysplasia prior to the development of cancer. However, colonoscopy is an invasive procedure and some individuals develop CRC despite undergoing repeated colonoscopic procedures. MicroRNAs (miRNAs) are small highly conserved non-coding single stranded RNA molecules. miRNAs are known to regulate gene expression and have been demonstrated to play an important role in cell proliferation, apoptosis and differentiation. Their main function is to down regulate gene expression. A growing number of studies have shown that miRNAs are involved in cancer. miRNAs have been found to be differently expressed in normal and tumor tissues. Indeed, more than 50% of human miRNA genes are located at sites known to be involved in cancers, suggesting their role in tumorigenesis. Due to their great stability, miRNAs can be easily isolated from tissue and from blood. A large number of mature miRNAs have been isolated from human colorectal cell lines, suggesting their value in the diagnosis of CRC. In this pilot study, we assess if miRNAs isolated from the serum of patients with IBD can identify those individuals with established colorectal neoplasia or those at risk of developing colorectal neoplasia. In addition, we compare miRNA profiles in serum from patients with IBD-associated colorectal neoplasia to non-IBD patients found to have sporadic CRC.

Patients participating in the study are selected from the following groups: (1) patients with IBD undergoing colonoscopy for colorectal neoplasia surveillance (40 patients selected); (2) patients with IBD with known colorectal neoplasia undergoing a colectomy (15 patients selected); (3) non-IBD patients with known colorectal neoplasia and are undergoing a colectomy (15 patients selected). Patients unable to give informed consent are excluded from the study.

We enroll n=40 patients at high risk of developing colorectal neoplasia, measure miRNA data at enrollment, and then follow for 2 years for the diagnosis of colorectal neoplasia. We expect between 5 and 10 of these patients to develop colorectal neoplasia over the study period. In addition, we obtain blood samples from IBD patients with known colorectal neoplasia (15) undergoing colectomy and another group of non-IBD patients with colorectal neoplasia (15) undergoing colectomy and compare these results to our cohort of 40 IBD patients at high risk of developing colorectal neoplasia.

The subject gives a blood sample prior to his/her colonoscopy or colectomy. This sample is collected prior to the procedure by a nurse either at the time of IV placement for the colonoscopy or in the office, preferably during a routine blood draw. For subjects who have an annual colonoscopy, their blood is drawn at the procedure for the next 1-3 years, unless they withdraw. For subjects having a colectomy, the study duration is the single pre-operative blood draw.

The blood sample is labeled with the subject's unique study identification number and is packaged and shipped the same day to the testing site in shipping kits in accordance with federal regulations pertaining to shipping biologicals. The sample is processed, analyzed for microRNA profiles and then stored until the completion of the study in the event that the samples need to be retested for microRNA profiles.

MicroRNA levels are expected to be highly skewed, and analyses will be on log transformed values or through non-parametric methods, depending on the amount of skewness observed in the data. Correlations between microRNA markers are explored through correlation (Pearson's or Spearman's depending on the distribution of the data). MicroRNA levels are compared between patients developing cancer vs. those not developing cancer through the two-sample t-test or Wilcoxon rank sum test. Optimal cut-points for screening values for microRNA levels are examined through ROC curve analysis, and sensitivity, specificity, and positive predictive value for different screening rules are described.

MicroRNA results are sent to the investigator. The investigator reviews the patient's medical record for the following information: age, gender, race, smoking/non-smoking, allergies, family history of IBD, family history of colon cancer, year of IBD diagnosis, extent and severity of IBD, IBD surgery history, current medication list and previous history of anti-TNFs, 6 MP, azathioprine, methotrexate. The investigator enters the data into the study database, matching it with the unique identification number. The lab results from the testing site are also added to the database upon receipt. Upon completion of the colonoscopy or colectomy, the investigator reviews the pathology results in the patient's medical record and then enters the results into the de-identified database.

In this pilot study, we assess if miRNA isolated from the serum of patients with IBD can identify those individuals with established colorectal neoplasia or those at risk of developing colorectal neoplasia. In addition, we compare miRNA profiles in serum from patients with IBD-associated colorectal neoplasia to non-IBD patients found to have sporadic CRC.

A study comparing miRNA data for colorectal cancer patients vs. healthy control reported a 4.5-fold increase in miR-92 in a preliminary sample of 5 cancer and 5 control patients, and confirmed this difference (corresponding to an estimated effect size for log miR-92 of 1.1) in samples of 25 cancer and 20 control patients (Ng et al., Differential expression of miRNAs in plasma of patients with colorectal cancer: a potential marker for colorectal cancer screening, Gut 2009). If the differences observed between cancer patients and controls hold for our sample of pre-cancer patients and controls, our study has between 62% power (if our sample yields 5/50 colorectal neoplasia patients) and 86% power (if our sample yields 10/50 colorectal neoplasia patients) of showing this difference at the two-tailed p<0.05 level. Estimates of sensitivity, specificity, and positive predictive value may be determined for a screen based on miRNA data. If 5/5 colorectal neoplasia patients were detected by this screen (100% sensitivity), the 95% lower confidence limit for sensitivity would be 55%. If 41/45 non-cancer patients screened negative (90% specificity), the 95% lower confidence limit for specificity would be 81%.

### Example 2

### Blood microRNA (miRNA) Expression Patterns can Identify Inflammatory Bowel Disease (IBD) Patients with Dysplasia or Cancer

This example describes one embodiment of a method for identifying microRNA expression patterns in inflammatory bowel disease (IBD) patients with dysplasia or cancer. Patients with long-standing and extensive IBD have an increased risk of developing dysplasia (D) or cancer (C). MicroRNAs (miRNAs) are a class of small non-coding RNA involved in post-transcriptional regulation of gene expression and have been demonstrated to play important roles in cell proliferation, apoptosis and differentiation. miRNAs have been found to be differently expressed in normal and tumor tissues. This example describes a study that categorizes serum miRNA profiles in IBD patients with dysplasia or cancer.

IBD patients (2 with IBD/D, 2 with IBD/C, 6 IBD controls without D/C) and 2 non-IBD sporadic C patients were identified at the Boston Medical Center IBD Center. miRNA were extracted from blood using Qiagen PAXgene miRNA kit. MiRNA from 2 patients with IBD/D, 2 with IBD/C, 6 IBD controls (without D/C) and 2 with non-IBD sporadic C were screened using the SmartChip Human MicroRNA Panel (Wafergen Biosystems) that consist of 1200 microRNA specific assays. A pool of 15 positive miRNAs down-regulated at least two fold was selected for additional study; every assay was subsequently confirmed using individual TaqMan miRNA assays (Applied Biosystems). The data have been normalized using U6 snRNA.

A set of 15 miRNA were identified as down-regulated in the IBD/D and IBD/C patients: hsa-miR-16, hsa-miR-15b, hsa-miR-15a, hsa-let-7f, hsa-miR-30b, hsa-miR-649, hsa-miR-575, hsa-miR-106a, hsa-let-7g, hsa-miR-223, hsa-miR-17, hsa-miR-652, hsa-miR-1287, hsa-miR-1229, and hsa-miR-21. To assess the specificity of this 15 miRNA pool, an additional 12 IBD controls were tested. Six IBD samples showed no expression differences while the remaining 6 samples had only 1 out of the 15 miRNA down-regulated. No difference was observed between UC and CD patients. The two non-IBD sporadic colorectal cancer patients used in the initial screening studies were confirmed to have no decrease in expression of these 15 miRNAs. Six samples from four IBD/D patients that include two samples initially screened were also tested with the 15 miRNA set. Three of these samples showed a difference of expression profile for at least 3 miRNAs. In IBD/C patients, 2 of 3 samples tested were also identified as having 3 or more miRNAs down-regulated.

We have identified a pool of 15 miRNAs that can distinguish between IBD patients with and without dysplasia or cancer. None of the IBD control patients displayed more than one down-regulated miRNA. These data indicate that when using this unique set of 15 miRNAs, patients with 3 or more down-regulated miRNAs should be suspected as having dysplasia or cancer.

### Example 3

### Blood microRNA Expression Patterns can Identify Inflammatory Bowel Disease Patients with Dysplasia or Cancer

This example describes another embodiment of a method for identifying microRNA expression patterns in inflammatory bowel disease (IBD) patients with dysplasia or cancer.

### Background

Patients with long standing and extensive IBD have an increased risk of developing dysplasia and cancer. Colonoscopic surveillance entails the risks and inconvenience of colonoscopy and needs to be repeated regularly. Interval cancers can develop despite appropriate surveillance protocols. Non-colonoscopic methods for surveillance are needed.

MicroRNAs (miRNAs) are a class of small non-coding RNAs involved in post-transcriptional regulation of gene expression that play important roles in cell proliferation, apoptosis and differentiation. More than 50% of human miRNA genes are located at sites involved in cancer, suggesting a role in tumorigenesis. miRNAs have been found to be differently expressed in normal and tumor tissues. Recent studies have identified circulating miRNAs in patients with digestive tract cancers.

Unique miRNA expression profiles are present in colonic mucosal biopsies from patients with IBD and dysplasia. Altered expression profiles are present in epithelial cells and peripheral blood of patients with active and inactive IBD compared with non-IBD controls. However, there are no data on miRNA profiles in serum or peripheral blood mononuclear cells in patients with IBD and dysplasia or cancer.

### Purpose

The aim of this study is to categorize circulating whole blood miRNA profiles in IBD patients with and without dysplasia or cancer.

### Methods

Blood samples were obtained in PAXGene solution from patients with extensive and longstanding UC or CD and used as a training set to identify differentially expressed miRNAs:
2 patients with IBD and dysplasia
2 patients with IBD and cancer
6 IBD control patients without dysplasia or cancer
2 patients with non-IBD related sporadic CRC
All histology reviewed by an expert GI pathologist.

These samples were examined using a SmartChip human microRNA panel consisting of 1200 microRNA specific assays.

The “hits” were determined for their ability to differentiate IBD patients with and without flat dysplasia or cancer (with a minimum of a 2 fold change). After identification of the abnormally expressed miRNA, all the assays were performed using individual TaqMan assays. Gold standard was histologic interpretation.

RNU6B was used as endogenous controls. The results were confirmed using RNU48 as a reference as well. Fold change calculation was used to measure change in the expression level of the miRNA:
ΔCt=Ct(miRNA)-Ct(RNU6B)
ΔΔCt=ΔCt (sample)-ΔCt (IBD control)
Fold change: (2̂-ΔΔCt)
Ct=Threshold cycle.

### Results

Table 3 provides a list of the 14 individual miRNAs with at least a two-fold change in expression level.

TABLE 3
A set of 14 miRNA were identified as dysregulated in IBD/D and IBD/C patients.

| Gene ID | Sequence | miRBase Accession | Genomic Location |
|---|---|---|---|
| hsa-miR-30b | UGUAAACAUCCUACACUCAGCU | MIMAT0000420 | Chr.: 8; Loc: q24.22 |
| hsa-miR-106a | AAAAGUGCUUACAGUGCAGGUAG | MIMAT0000103 | Chr.: X; Loc: q26.2 |
| hsa-miR-15b | UAGCAGCACAUCAUGGUUUACA | MIMAT0000417 | Chr.: 3; Loc: q25.33 |
| hsa-miR-649 | AAACCUGUGUUGUUCAAGAGUC | MIMAT0003319 | Chr.: 22; Loc: q11.21 |
| hsa-miR-16 | UAGCAGCACGUAAAUAUUGGCG | MIMAT0000069 | Chr.: 13; Loc: q14.2 |
| hsa-miR-652 | AAUGGCGCCACUAGGGUUGUG | MIMAT0003322 | Chr.: X; Loc: q23 |
| hsa-let-7f | UGAGGUAGUAGAUUGUAUAGUU | MIMAT0000067 | Chr.: 9; Loc: q22.32 |
| hsa-let-7g | UGAGGUAGUAGUUUGUACAGUU | MIMAT0000414 | Chr.: 3; Loc: p21.1 |
| hsa-miR-1287 | UGCUGGAUCAGUGGUUCGAGUC | MIMAT0005878 | Chr.: 10; Loc: q24.2 |
| hsa-miR-17 | CAAAGUGCUUACAGUGCAGGUAG | MIMAT0000070 | Chr.: 13; Loc: q31.3 |
| hsa-miR-1229 | CUCUCACCACUGCCCUCCCACAG | MIMAT0005584 | Chr.: 5; Loc: q35.3 |
| hsa-miR-28-3p | CACUAGAUUGUGAGCUCCUGGA | MIMAT0004502 | Chr.: 3; Loc: q28 |
| hsa-miR-182 | UUUGGCAAUGGUAGAACUCACACU | MIMAT0000259 | Chr.: 7; Loc: q32.2 |
| hsa-let-7g* | CUGUACAGGCCACUGCCUUGC | MIMAT0004584 | Chr.: 3; Loc: p21.1 |

Table 4 provides possible roles and targets for each of these 14 individual miRNAs.

TABLE 4

| Gene ID | Possible Roles/Targets |
|---|---|
| hsa-miR-30b | LIN28B: promotes cell migration, invasion and transformation, post-transcriptional |
| | regulation of LGR5 and PROM1 |
| hsa-miR-106a | miR-106a can increase p53 expression via E2F1 inhibition; E2F1: can mediate both cell |
| | proliferation and p53-dependent apoptosis |
| hsa-miR-15b | down-regulated expression induced by hypoxia in CRC suppresses enhances SMURF1/2 |
| | expression through SMAD7 to degrade TGFBR1/2 and inhibit SMAD2/3 & SMAD4 |
| | function |
| hsa-miR-649 | SPRY4: Suppresses the insulin receptor and EGFR-transduced MAPK signaling pathway, |
| | but does not inhibit MAPK activation by a constitutively active mutant Ras. Probably |
| | impairs the formation of GTP-Ras |
| hsa-miR-16 | miR-16 plays a role in COX-2 mRNA destabilization and suppresses BCL-2 |
| hsa-miR-652 | Inhibits AKT; GABBR1: the levels of GABA receptors or other signaling components are |
| | up-regulated in cancer cells |
| hsa-let-7f | MAPK8, TGFBR1, IGF1R, TP53, ACVR1C, CCND1 |
| hsa-let-7g | may act as a tumor suppressor gene by down-regulating the oncogene, c-Myc, and up- |
| | regulating the tumor suppressor gene, MTS1 |
| hsa-miR-1287 | IER-3: May play a role in the ERK signaling pathway by inhibiting the dephosphorylation |
| | of ERK by phosphatase PP2A-PPP2R5C holoenzyme |
| hsa-miR-17 | JAK1: Expressed at higher levels in primary colon tumors than in normal colon tissue. The |
| | expression level in metastatic colon tumors is comparable to the expression level in normal |
| | colon tissue |
| hsa-miR-1229 | Part of a cytoplasmic complex made of HIPK1, DAB2IP and MAP3K5 in response to TNF. |
| | This complex formation promotes MAP3K5-JNK activation and subsequent apoptosis. |
| hsa-miR-28-3p | CXXC5 [symbol] May indirectly participate in activation of the NF-kappa-B and MAPK |
| | pathways. Acts as a mediator of BMP4-mediated modulation of canonical Wnt signaling |
| | activity [symbol] AXIN2 = down-regulation of beta-catenin |
| hsa-miR-182 | MTSS1: May be related to cancer progression or tumor metastasis in a variety of organ |
| | sites, most likely through an interaction with the actin cytoskeleton |
| hsa-let-7g* | suppressing HNF4A expression; ZEB2: represses transcription of e-Cadherin |

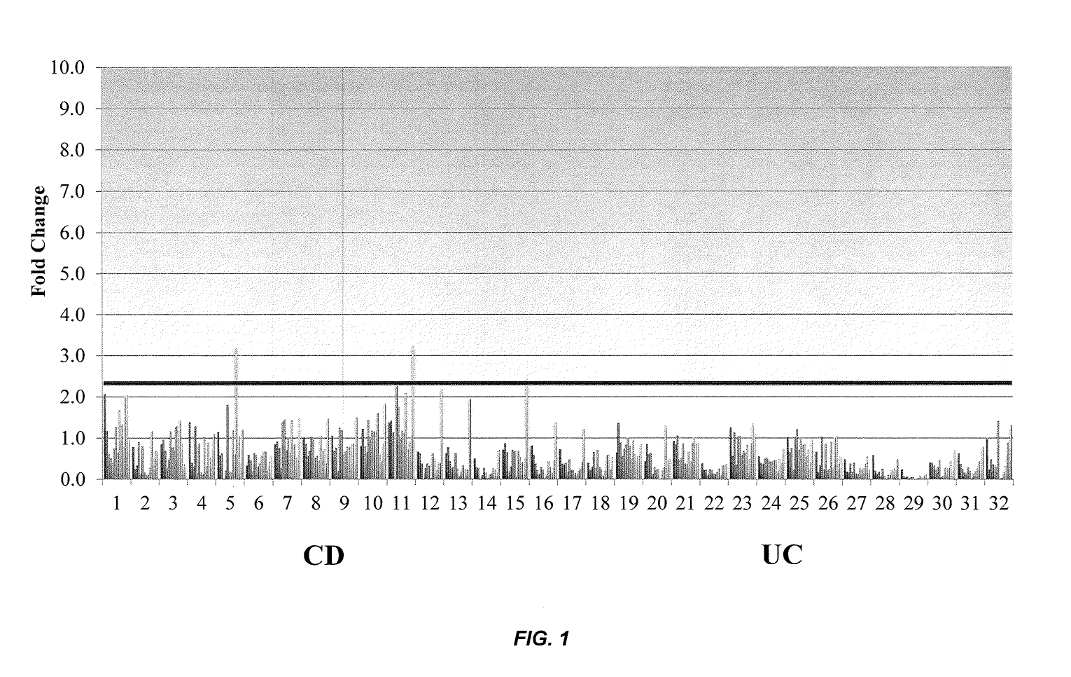

For FIGS. 1-6, each vertical bar for a given sample corresponds to the following miRNAs (from left to right): hsa-miR-30b, hsa-miR-106a, hsa-miR-15b, hsa-miR-649, hsa-miR-16, hsa-miR-652, hsa-let-7f, hsa-let-7g, hsa-miR-1287, hsa-miR-17, hsa-miR-1229, hsa-miR-28-3p, hsa-miR-182, hsa-let-7g*.

FIG. 1 shows archived IBD samples (CD or UC) without dysplasia or cancer. To differentiate the disease samples from the IBD controls, an arbitrary cut-off of three or more miRNA with at least 2.3 fold change was chosen.

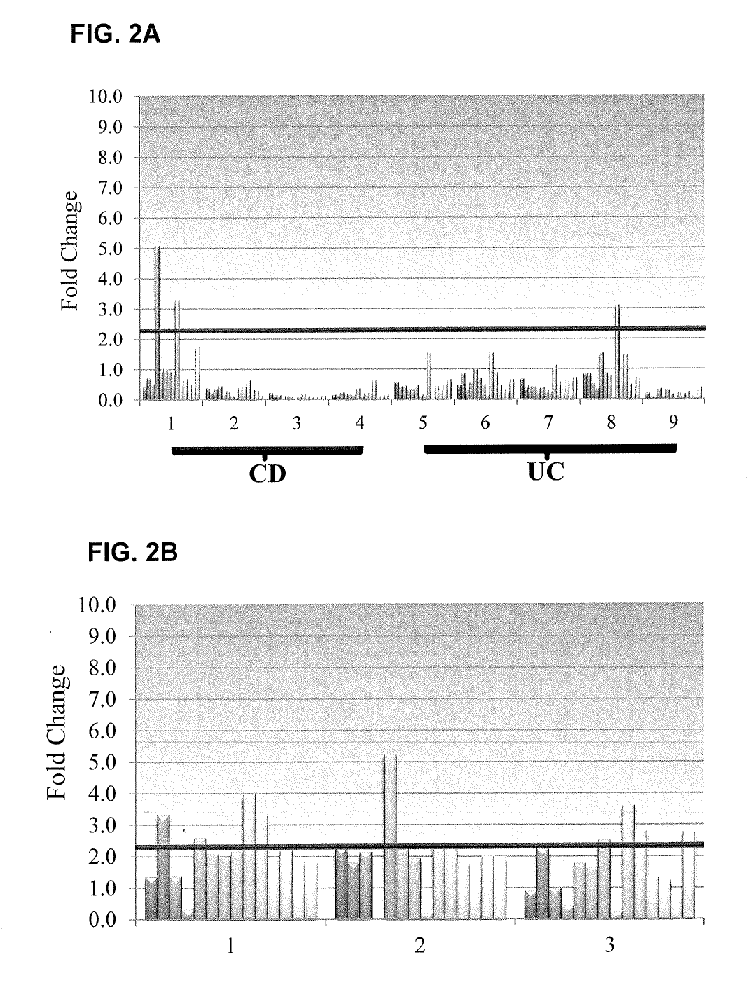

FIG. 2A shows nine control IBD samples that were negative using the miRNA assay. FIG. 2B shows three control IBD samples that were positive using the miRNA assay. 1: UC; no dysplasia; repeat colonoscopy pending. 2: CD ileocolonic; no dysplasia, but patient with concomitant hyperplastic polyposis. 3: UC; no dysplasia, but multiple inflammatory polyps; repeat colonoscopy pending.

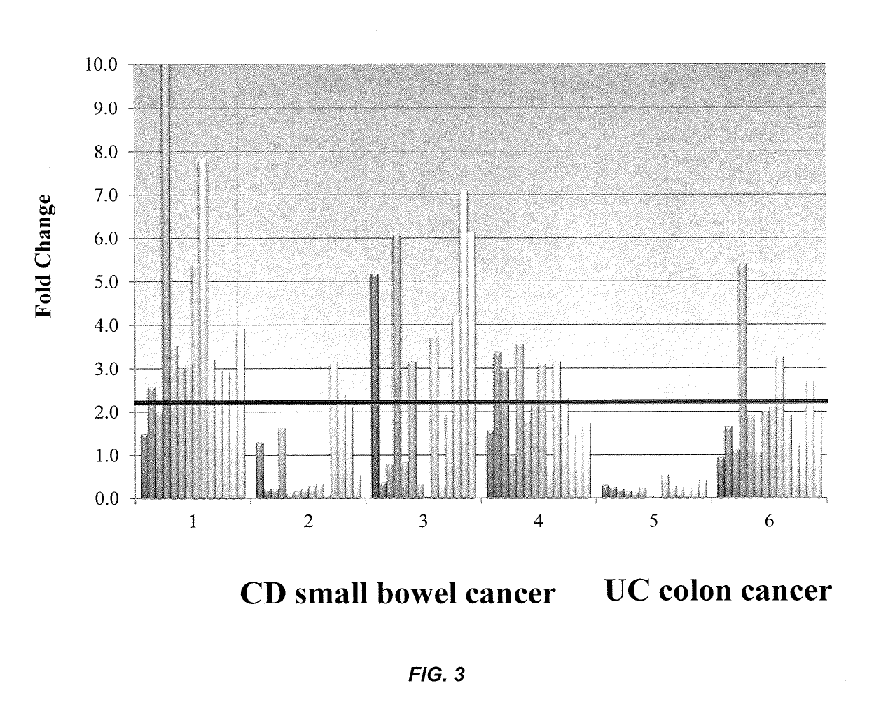

FIG. 3 shows that the set of 14 miRNAs described herein can identify IBD patients with cancer. Patients 1, 2, 3, and 4 have CD with small bowel cancer. Patients 5 and 6 have UC with colon cancer.

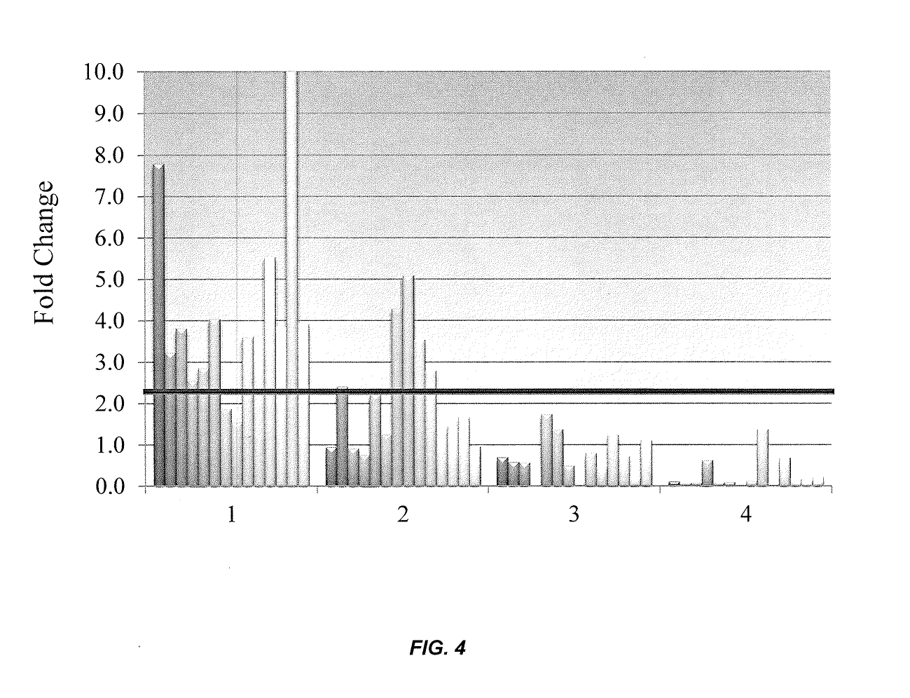

FIG. 4 shows that the set of 14 miRNAs described herein can identify IBD patients with flat dysplasia. 1: UC dysplasia. 2: IBD indefinite dysplasia. 3: CD-(low grade dysplasia); repeat colonoscopy pending. 4: UC and primary sclerosing cholangitis with indefinite dysplasia; repeat colonoscopy pending.

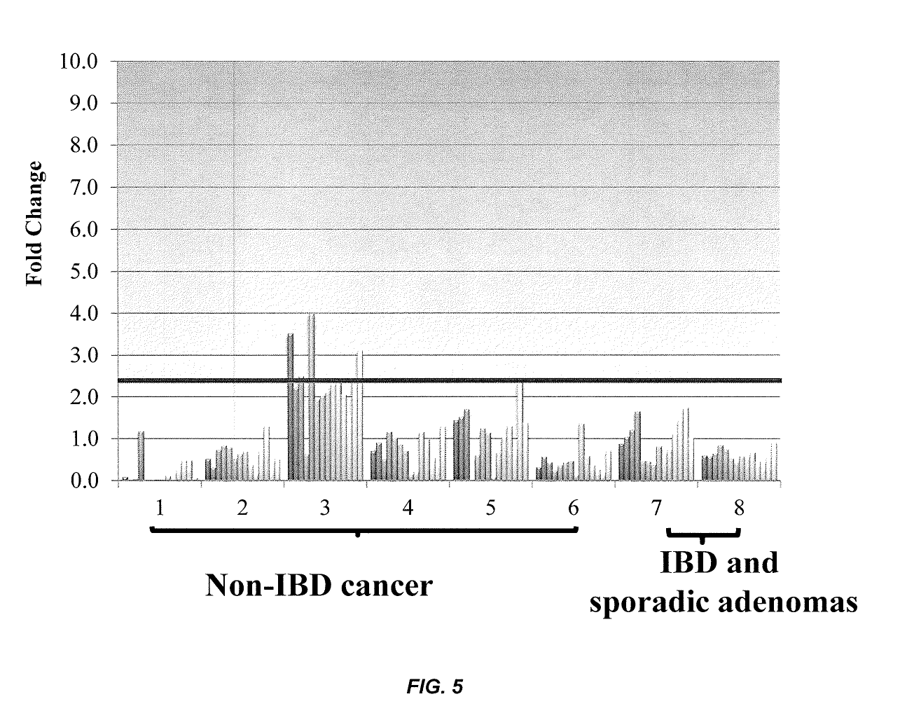

FIG. 5 shows non-IBD colon cancer and sporadic tubular adenomas in IBD. Only non-IBD colorectal cancer patient #3 was positive.

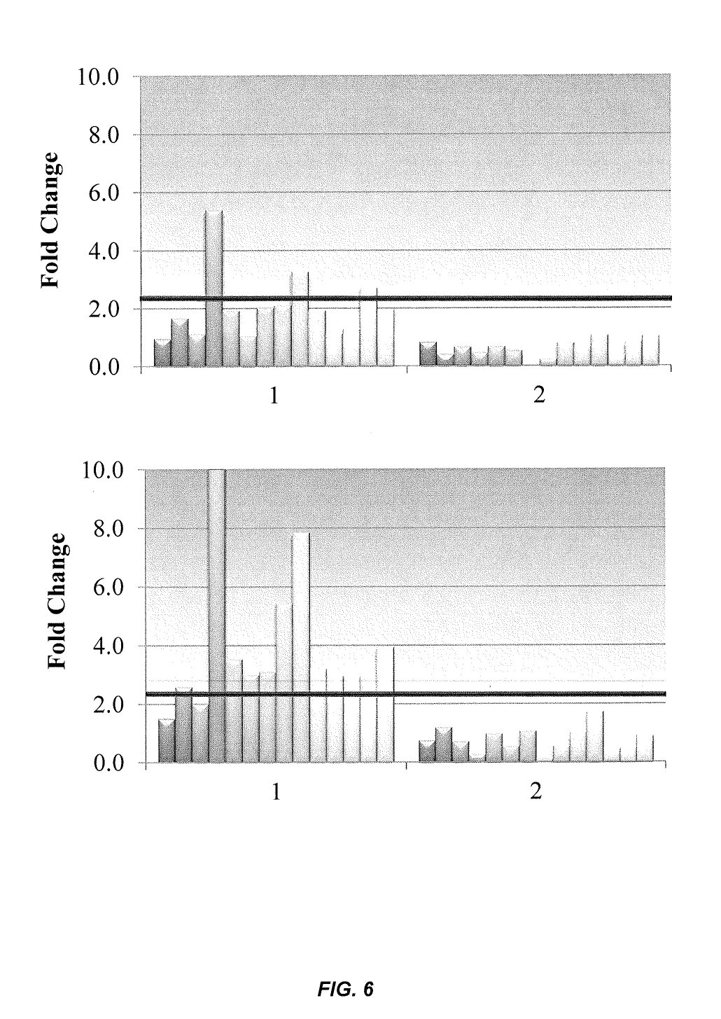

FIG. 6 shows that the set of 14 miRNAs described herein can be used to monitor CD patients with small bowel cancer before and after treatment. 1: Pre-operative. 2: Post-surgery and chemotherapy and in remission. Two different CD patients with small bowel cancer were monitored.

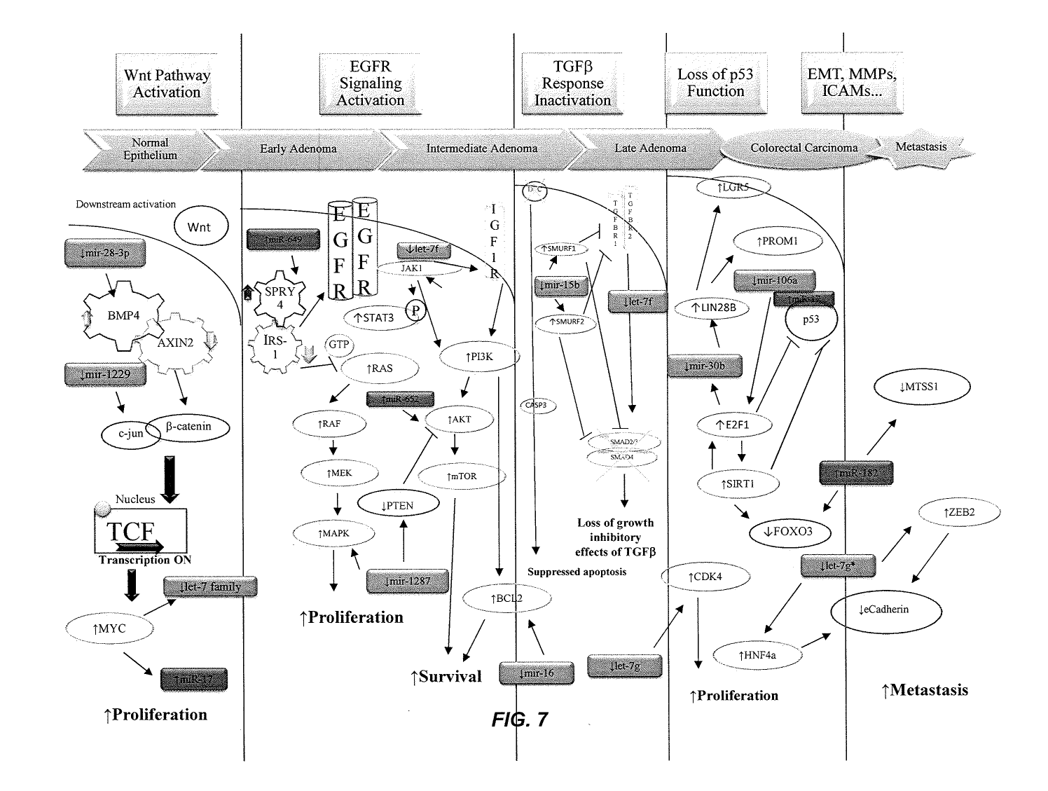

FIG. 7 shows one embodiment of a pathway analysis for the pool of 14 miRNAs identified as being dysregulated in IBD patients with dysplasia or cancer. For example, this figure shows the direction of the change in the level of expression for each miRNA or family of miRNAs, and also illustrates the signaling pathway(s) and/or stage(s) of colorectal cancer (CRC) progression and metastasis that the dysregulation of these miRNAs play a role.

### SUMMARY

In these studies, we have identified a pool of 14 miRNAs that can identify IBD patients with dysplasia or cancer. Three of 45 IBD control patients displayed an abnormal profile; repeat colonoscopy pending in these 3 patients. Sporadic adenomas in 2 IBD patients were not detected using these miRNA profiles. These data indicate that when using this set of 14 miRNA, IBD patients with 3 or more dysregulated miRNAs should be suspected of having IBD-related dysplasia or cancer.

### REFERENCES

Farraye F A, et al., Gastroenterology. 2010 February; 138(2):746-774.
Ichikawa D, et al. Gastroenterology. 2012 May; 142(5):1074-1078.
Zhu et al. BMC Research Notes 2009, 2, 89.
Bushati N, et al. Annu. Rev Cell Dev Biol. 2007. 23:175-205.
Dalai S R, et al. Gastroenterol Hepatol (NY). 2010 November; 6(11):714-22.
Pekow J R, et al. Inflamm Bowel Dis. 2012 January; 18(1):187-93.

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A method for identifying an inflammatory bowel disease (IBD) patient with dysplasia or cancer, the method comprising:
(a) determining the presence or level of at least one or a plurality of miRNAs in a sample from the patient to establish a miRNA expression profile;
(b) comparing the miRNA expression profile to one or more pre-established model miRNA expression profiles from a control and/or reference standard; and
(c) identifying dysplasia or cancer in the IBD patient based upon the comparison in step (b).

2. The method of claim 1, wherein the plurality of miRNAs comprises at least 2, 3, 4, 5, 6, 7, 8, 9, 10, 11, 12, 13, 14, or 15 miRNAs.

3. The method of claim 1, wherein at least one or a plurality of the miRNAs in the miRNA expression profile is dysregulated compared to the identical miRNA in the one or more pre-established model miRNA expression profiles.

4. The method of claim 3, wherein a miRNA in the miRNA expression profile is dysregulated when there is at least about a 2-fold change in the expression level of the miRNA compared to the identical miRNA in the one or more pre-established model miRNA expression profiles.

5. The method of claim 3, wherein the IBD patient is identified as having or suspected of having dysplasia or cancer when at least 3 miRNAs in the miRNA expression profile are dysregulated compared to the identical miRNAs in the one or more pre-established model miRNA expression profiles.

6. The method of claim 1, wherein the control and/or reference standard is selected from the group consisting of an IBD control sample without dysplasia or cancer, a non-IBD cancer sample, and combinations thereof.

7. The method of claim 1, wherein the at least one or a plurality of miRNAs is selected from the group consisting of hsa-miR-30b, hsa-miR-106a, hsa-miR-15b, hsa-miR-649, hsa-miR-16, hsa-miR-652, hsa-let-7f, hsa-let-7g, hsa-miR-1287, hsa-miR-17, hsa-miR-1229, hsa-miR-28-3p, hsa-miR-182, hsa-let-7g*, and combinations thereof.

8. The method of claim 7, wherein the presence or level of at least 1, 2, 3, 4, 5, 6, 7, 8, 9, 10, 11, 12, 13, or all 14 of the miRNAs are determined to establish the miRNA expression profile.

9. The method of claim 1, wherein the sample is whole blood, serum, or plasma.

10. The method of claim 1, wherein the IBD patient is a Crohn's disease (CD) patient or an ulcerative colitis (UC) patient.

11. The method of claim 1, wherein the dysplasia is selected from the group consisting of flat dysplasia, elevated dysplasia, and indefinite dysplasia.

12. The method of claim 1, wherein the cancer is selected from the group consisting of small bowel cancer, colorectal cancer, and combinations thereof.

13. A method for monitoring the efficacy of treatment of an IBD patient with dysplasia or cancer, the method comprising:
(a) determining the presence or level of at least one or a plurality of miRNAs in a first sample from the patient to establish a first miRNA expression profile, wherein the first sample is obtained before treatment;
(b) determining the presence or level of the identical miRNAs in a second sample from the patient to establish a second miRNA expression profile, wherein the second sample is obtained during or after treatment;
(c) comparing the first miRNA expression profile to the second miRNA expression profile; and
(d) monitoring the efficacy of treatment for the IBD patient based upon the comparison in step (c).

14. The method of claim 13, wherein the plurality of miRNAs comprises at least 2, 3, 4, 5, 6, 7, 8, 9, 10, 11, 12, 13, 14, or 15 miRNAs.

15. The method of claim 13, further comprising comparing the first and/or second miRNA expression profile with one or more pre-established model miRNA expression profiles from a control and/or reference standard.

16. The method of claim 15, wherein the control and/or reference standard is selected from the group consisting of an IBD control sample without dysplasia or cancer, a non-IBD cancer sample, and combinations thereof.

17. The method of claim 15, wherein at least one or a plurality of the miRNAs in the first and/or second miRNA expression profile is dysregulated compared to the identical miRNA in the one or more pre-established model miRNA expression profiles.

18. The method of claim 17, wherein a miRNA in the first and/or second miRNA expression profile is dysregulated when there is at least about a 2-fold change in the expression level of the miRNA compared to the identical miRNA in the one or more pre-established model miRNA expression profiles.

19. The method of claim 17, wherein the IBD patient is responsive to treatment when at least 3 miRNAs are dysregulated in the first miRNA expression profile and the identical miRNAs are not dysregulated in the second miRNA expression profile.

20. The method of claim 17, wherein the IBD patient is unresponsive to treatment when at least 3 miRNAs are dysregulated in the first miRNA expression profile and the identical miRNAs are dysregulated in the second miRNA expression profile.

21. The method of claim 13, wherein the treatment is selected from the group consisting of chemotherapy, surgery, and combinations thereof.

22. The method of claim 13, wherein the at least one or a plurality of miRNAs is selected from the group consisting of hsa-miR-30b, hsa-miR-106a, hsa-miR-15b, hsa-miR-649, hsa-miR-16, hsa-miR-652, hsa-let-7f, hsa-let-7g, hsa-miR-1287, hsa-miR-17, hsa-miR-1229, hsa-miR-28-3p, hsa-miR-182, hsa-let-7g*, and combinations thereof.

23. The method of claim 22, wherein the presence or level of at least 1, 2, 3, 4, 5, 6, 7, 8, 9, 10, 11, 12, 13, or all 14 of the miRNAs are determined to establish the first and second miRNA expression profiles.

24. The method of claim 13, wherein the first and second samples are independently selected from the group consisting of whole blood, serum, and plasma.

25. The method of claim 13, wherein the IBD patient is a Crohn's disease (CD) patient or an ulcerative colitis (UC) patient.

26. The method of claim 13, wherein the dysplasia is selected from the group consisting of flat dysplasia, elevated dysplasia, and indefinite dysplasia.

27. The method of claim 13, wherein the cancer is selected from the group consisting of small bowel cancer, colorectal cancer, and combinations thereof.
